(19) United States
(12) Patent Application Publication
(10) Pub. No.: US 2004/0045041 A1
(43) Pub. Date: Mar. 4, 2004

(54) Means for the identification compounds capable of inhibiting karap-transduced signals

(76) Inventors: Eric Vivier, Cassis (FR); Frederic Vely, Cassis (FR); Elena Tomasello, Marseille (FR)

Correspondence Address: NIXON & VANDERHYE, PC, 1100 N GLEBE ROAD, 8TH FLOOR, ARLINGTON, VA, 22201-4714, US

(21) Appl. No.: 10/381,018

(22) PCT Filed: Sep. 20, 2001
(86) PCT No.: PCT/EP01/11492

(30) Foreign Application Priority Data
Sep. 20, 2000 (US) 60234161

(51) Int. Cl. G01N 33/00

(52) U.S. Cl. 800/3

(57) ABSTRACT

The present application relates to transgenic animals over-expressing KARAP, knock-in animals bearing non functional KARAP, and to method and kits for the identification of compounds capable of inhibiting a KARAP-transduced immune signal.

The invention relates to means for the identification of compounds capable of inhibiting KARAP-transduced signals.

KARAP (KAR-Associated Proteins) have already been reported, and fully described in WO 98/49292 in the name of I.N.S.E.R.M. of which content is herewith fully incorporated by reference. KARAP have also been referred to as DAP12 (Lanier et al. Feb. 12, 1998, Nature vol.39: 703-707). WO 98/49292 namely describes several embodiments enabling the isolation of KARAP, and gives illustrative human and mouse KARAP sequences. More particularly, KARAP polypeptides are known to associate with KAR (also referred to as KIR-S) and with KAR-alike receptors, and to be necessary for transducing a signal originating from such receptors. More particularly, KARAP is now known to associate with KAR (NKG2C, p50.2) and NKp44 in NK cells, with TREM1, TREM2 (myeloid cells in general, and more particularly dendritic cells, macrophages), and with SIRPβ present in a wide variety of cells of hematopoietic and non hematopoietic origin.

The inventors have produced cells co-expressing a functional KARAP and a functional receptor which transduces its signal via ζ, γ or ε, transgenic cells over-expressing functional KARAP, and transgenic animals of which cells over-express KARAP. They have also produced non functional. KARAP, knock-in cells bearing non functional KARAP, and knock-in animals bearing non functional KARAP. Illustrative methods for producing such products are described in the below examples.

The experimental results obtained with these materials directly confirm the in vivo involvement of KARAP in the control of the immune system responses, directly confirm that this involvement is not restricted to NK cells and KAR, but also concerns other KAR-alike activatory receptors and other cells such as myeloid cells and in particular dendritic cells (DC). These results also directly confirms the in vivo involvement of KARAP in the control of immune responses, and in particular in the control of tumor development, allergy and auto-immune diseases such as contact sensitivity and multiple sclerosis. These in vivo results altogether further on support the utility of KARAP compounds and of KARAP-inhibiting compounds in the production of drugs intended for the prevention, therapy or palliation of undesired immune responses. KARAP compounds stimulate the immune response of cells such as NK cells and myeloid cells (dendritic cells, macrophages in particular), such as e.g. the lytic activity of NK cells towards tumor cells, whereas KARAP-inhibiting compounds inhibit the immune response of these cells.

The inventors have therefore now developed tools for the development of KARAP-inhibiting compounds. Such KARAP-inhibiting compounds are very useful as active agents in the formulation of drugs intended for inhibiting undesired immune responses, and in particular for inhibiting the activity of cells favoring auto-immune or allo-immune reactions (more particularly, multiple sclerosis, graft rejection, and allergic reactions such as contact sensitivity). They have also demonstrated that KARAP-inhibiting compounds impair the development and maturation of dendritic cells (they inhibit antigenic presentation of dendritic cells, either directly via synthesis inhibition or through inhibition of the migration of dendritic cells).

Besides from their capacity for inhibiting NK cell activity, such KARAP-inhibiting compounds are thus very useful as active agents in the formulation of drugs intended for inhibiting dendritic cell development or maturation.

The tools of the invention are based on particular combined use of different techniques and products, such as notably functional KARAP, non functional KARAP, cells co-expressing a functional KARAP and a functional receptor which transduces its signal via ζ, γ or ε, engineered cells and animals over-expressing functional KARAP, engineered cells and animals bearing a non functional KARAP. These tools corresponds to methods and kits for the identification of compounds capable of inhibiting a KARAP-transduced signal.

These methods and kits have the particular advantage of enabling the identification of KARAP inhibitors with accurate KARAP specificity. They also have the advantage of easy applicability, and rapid and reliable performances.

A method of the invention notably comprises the administration of test compound to a transgenic animal that expresses functional KARAP in at least 3 copies, preferably more than 4 copies, and more preferably in 5, 11, or 30 copies, the test compound being selected as an inhibitor of KARAP-transduced signal when it significantly increases the life duration of the animal, and/or significantly increases the number of lymphoid cells in the animal, and/or significantly decreases the number of myeloid cells in the animal. This method will be referred to as the over-expression method of the invention.

According to another embodiment, a method of the invention comprises the administration of a test compound to an animal which expresses a normal number of copy of functional KARAP (i.e. 2 copies) and in which a reaction involving at least KARAP has been triggered, the test compound being selected as an inhibitor of KARAP-transduced signal when it significantly inhibits said reaction.

According to an improvement of this embodiment, a method of the invention comprises:

inducing a reaction involving at least KARAP, in animals which express a normal number of copy (i.e. 2 copies) of functional KARAP, these animals being referred to as normal animals,

applying the same reaction protocol to animals which have been engineered so as to bear non functional KARAP, these animals being referred to as knock-in animals,

applying the same reaction protocol to a transgenic animal that expresses functional KARAP in at least 3 copies, preferably more than 4 copies, and more preferably in 5, 11, or 30 copies,

administering the same test compound to the normal, knock-in and transgenic animals under same or comparable conditions, the test compound being selected as an inhibitor of KARAP-transduced signal when it inhibits said reaction of the normal animals, and when:

it does not significantly inhibit the reaction that may be observed in knock-in animals in response to said reaction protocol application (if any reaction is observed in these knock-in animals), and/or

it does inhibit said reaction in the normal animals to a level that is comparable to the one observed in the knock-in animals, and/or

it does inhibit said reaction in the transgenic animals,

it does not induce any significant immunological or non immunological side effects in the knock-in animals.

This method will be referred to as the KARAP-transduced reaction method of the invention.

Advantageous reactions involving at least KARAP are selected among the group consisting of contact sensitivity reaction and auto-immune disease. Appropriate methods to trigger a contact sensitivity reaction or an auto-immune disease in a normal animal are available to the skilled person. Illustrative methods are described in the examples below.

According to another embodiment, an alternative method is provided for the identification of compounds capable of inhibiting a KARAP-transduced immune response, this method comprising:

a) the bringing into contact of a test compound with a cell co-expressing i) a functional KARAP and a functional receptor that transduces signal through KARAP ii) a functional ζ or γ or ε, and a functional receptor that transduces signal through ζ or γ or ε,

b) the stimulation of this KARAP and this ζ or γ or ε-associated receptor, the test compound being selected as an inhibitor of KARAP-transduced signal when it significantly inhibits the KARAP-transduced signal and does not significantly inhibit the ζ or γ or ε-transduced signal.

Appropriate receptors which transduce their signal via ζ, γ or ε, notably include TcR receptor molecules and receptors having a high affinity with IgE such as FcγRI. Embodiments for stimulating an activatory receptor are widely known to the skilled person, and notably include cross-linking by antibodies. Appropriate conditions include conditions of the physiological type. Any cell fitting with the requirements recited in a) above is appropriate. Preferred cells are those which produces an easily-detectable signal when activated (e.g. production of serotonine); examples of such cells are given in the examples below. Advantageously, this method is combined with any one of the preceding methods, and is preferably performed prior to the steps of said preceding method(s) so as to allow a pre-selection of the test compounds (screening steps on cells).

Appropriate methods and tools for assessing the level of activation of a receptor, the level of a contact sensitivity reaction, the life duration of an animal, the viability of a cell are known to the skilled person. Illustrative procedures are given in the examples below. Appropriate methods for triggering a sensitivity reaction in an animal are available to the skilled person. Illustrative methods are described in the examples below. Appropriate method for producing engineered animals and cells either over-expressing functional KARAP or bearing non functional KARAP (knock-in animals and cells) are illustrated in the examples below. A method for producing transgenic animals over-expressing functional KARAP notably includes placing a wild type isolated KARAP sequence under control of a promoter which enables the in vivo expression of this KARAP sequence, such as e.g. H-2Kb promoter, and transfecting it in an animal oocyte. A method for producing knock-in animals notably includes homologous recombination of a non functional KARAP sequence (e.g. KARAP sequence deleted from at least one ITAM) with a functional KARAP sequence.

According to another embodiment, a method for the identification of compounds capable of inhibiting a KARAP-transduced signal without inhibiting another activatory molecules is provided. This method comprises the selection of those test compounds which interact with the charged. aminoacid of the KARAP molecule transmembrane region and/or with a charged aminoacid (K or R) which is centrally located within the transmembrane region of an activatory receptor. The respective positions of these charged aminoacids are indeed characteristic of KARAP molecules and of receptors that selectively associate with KARAP (see example 4 below). In the human KARAP molecule, this charged aminoacid is D in position 50, in the mouse KARAP, it is D in position 52. This method will be referred to as the charged aminoacid method of the invention. It indeed allows the selection of inhibiting compounds which interact with KARAP without interacting with other activatory molecules which are structurally very close to KARAP, such as e.g. CD3ζ, CD3γ, and FcγRI. This method can be advantageously combined with any one of the preceding methods. It is advantageously performed in combination with at least one of the preceding methods, and preferably prior to the steps of any one of the above methods.

A preferred method of the invention comprises the steps of the charged aminoacid method of the invention, followed by the steps of the alternative method of the invention (screening steps on cells), and then by the steps of an over-expression method of the invention and/or by the steps of a KARAP-transduced reaction method of the invention (screening on animals).

The present application also relates to kits for the implementation of a method intended for the identification of a compound which is capable of inhibiting a KARAP-transduced signal. The kits of the invention may comprise any combination of at least two elements selected from the group consisting of non functional KARAP, engineered cells co-expressing functional KARAP and ζ or γ or ε, animals bearing a normal copy number of functional KARAP, engineered animals bearing a number of KARAP copy equal to or above 3, engineered animals bearing non functional KARAP. The present application also individually describes any of these products. Kits comprising engineered animals bearing a normal copy number of functional KARAP may further comprise an agent capable of inducing a sensitivity contact reaction in an animal such as DNBF (2,4-dinitrofluorobenzene), or an agent capable of inducing an auto-immune disease such as pMOG peptide 33-55. Non functional KARAP notably include those KARAP wherein at least one ITAM motif has been deleted, and those KARAP wherein the Y residue of at least one of its ITAM motifs has been substituted by a phenylalanine, and those KARAP which have been chemically modified so as to prevent hydrolyzable phosphorylation on them.

Any animal may be appropriate in the present invention. For convenience purposes, non human mammals are preferred such as mice, rabbits, pigs. The inhibitory compounds identified by a method or a kit of the invention are useful for the treatment, prevention, palliation of immune response wherein KAR activation has to be inhibited. They are particularly appropriate in the case of contact sensitivity and multiple sclerosis.

The present results and invention are illustrated by the following examples which should be in no event considered as limitative. Further characteristics of the present invention can be found in these examples. The person of ordinary skill in the art can perform alternative embodiments without departing from the scope of the present results and application. Reference manuals give illustrative alternative embodiments to the skilled person, e.g. Maniatis, Molecular Cloning: A laboratory manual; Gene targeting: a practical approach (Practical approach series, 212), Alexandra L. Joyner (Editor), Oxford University Press ISBN019963792X ; Microinjection and transgenesis: strategies and protocol (Springer Lab Manual) Angel Cid-Arregui (Editor), Alejandro Garcia (Editor), Springer Verlag, ISBN350618953, of which contents are herewith incorporated by reference. Standard abbreviations have been used, such as mAb for monoclonal antibody(ies), and DC for dendritic cells.

These examples are illustrated by the following figures:

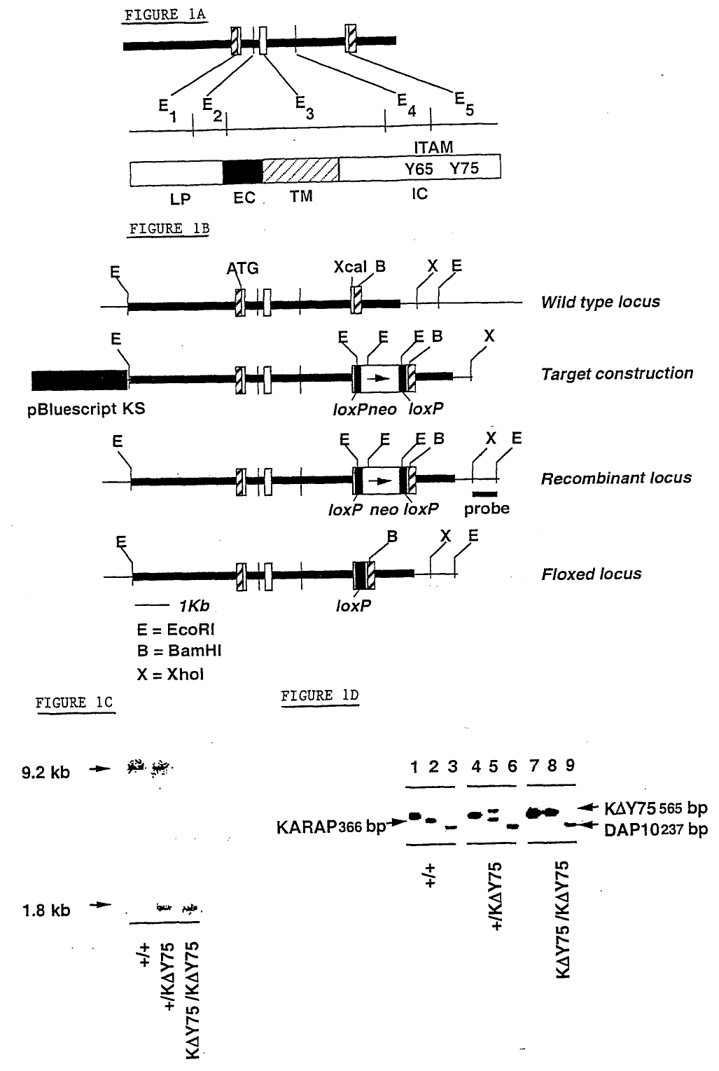

FIGS. 1A, 1B, 1C and 1D: Generation of KARAP/DAP12 knock-in mice (KΔY75/KΔY75 mice).

In FIG. 1A: the exon/intron organization of mouse KARAP/DAP12 gene in the 129 background is schematized (top; E: exon), and the corresponding KARAP/DAP12 protein is also represented (bottom; LP: leader peptide, EC: extracellular domain, TM: transmembrane domain, IC: intracellular domain). KARAP/DAP12 ITAM is centered on tyrosine residues Y65 and Y75, as indicated (shaded area).

In FIG. 1B: KARAP/DAP12 targeting strategy.

In FIG. 1C: Southern blot analysis. The 9.2 kb wild-type and the 1.8 kb targeted allele EcoRI-EcoRI fragments identified by probe E are indicated.

In FIG. 1D: RT-PCR analysis of +/+, +/KΔY75 and homozygous mutant (KΔY75/KΔY75) littermates.

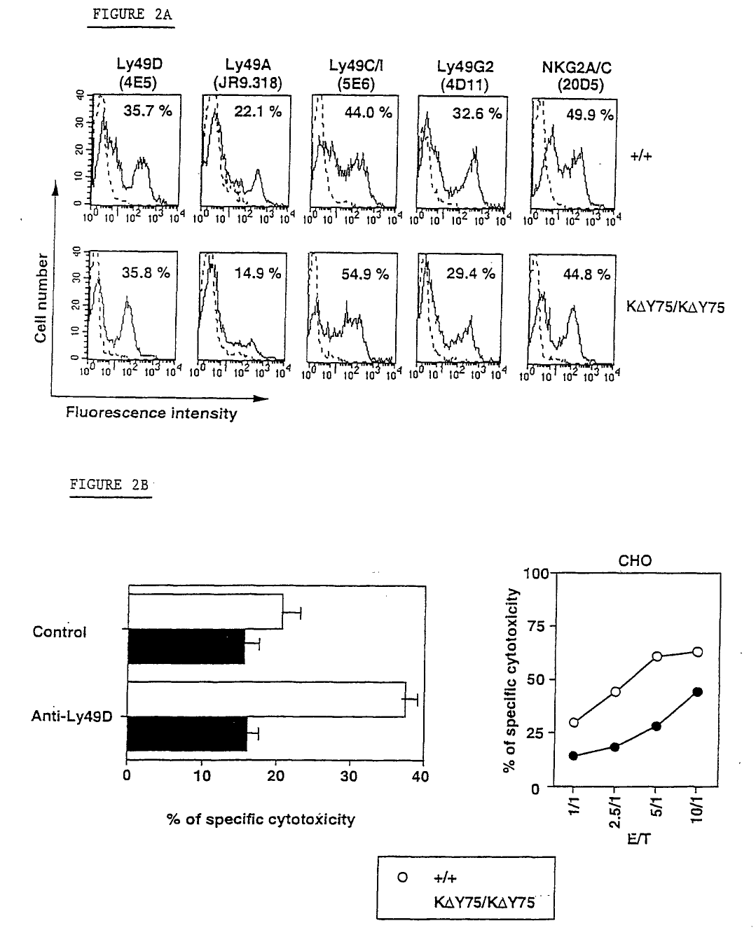

FIGS. 2A and 2B : Expression of activating and inhibitory MHC class I receptors on splenic NK cells isolated from control and KΔY75/KΔY75 mice.

In FIG. 2A: The cell surface expression of indicated receptors on CD3⁻ DX5⁺ splenic NK cells isolated from control mice (upper panels) and KΔY75/KΔY75 mice (lower panels) was analyzed by flow cytometry. The percentages of positive stained cells (continuous lines), as well as the control staining using isotype-matched control mAbs (dotted lines) are indicated.

In FIG. 2B:

Left panel: 7 days cultured IL-2 activated DX5⁺ splenocytes were analyzed in a 4 hr ⁵¹Cr release assay against the murine mastocytoma P815 (FcγR⁺) in the presence (anti-Ly49D) or absence (Control) of anti-Ly49D mAbs at the 2.5:1 E:T ratio. Control mice (open bars), KΔY75/KΔY75 mice (filled bars).

Right panel: 7 days cultured IL-2 activated DX5⁺ splenocytes were analyzed in a 4 hr ⁵¹Cr release assay against CHO tumor cell lines at the indicated E:T ratios. Control mice (open circles), KΔY75/KΔY75 mice (filled circles).

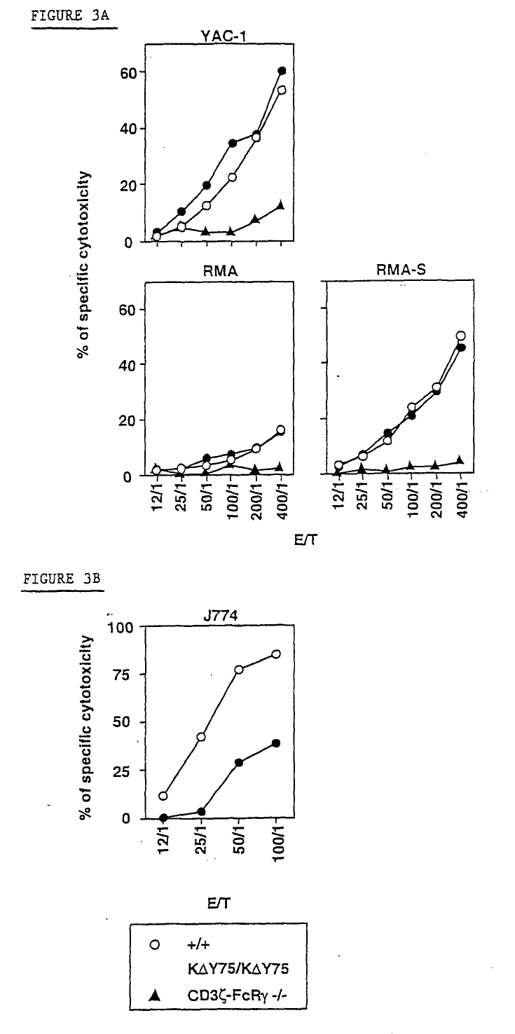

FIGS. 3A and 3B : Natural cytotoxicity exerted by NK cells isolated from control, KΔY75/KΔY75 and CD3ζ-FcRγ^{-/-} mice.

In FIG. 3A: Natural cytotoxicity exerted by splenocytes isolated from 8 hr poly-IC-treated mice was assessed in a standard 4 hr ⁵¹Cr release assay against indicated target cell lines. Control mice (open circles), KΔY75/KΔY75 mice (filled circles), CD3ζ-FcRγ^{-/-} mice (filled triangles).

In FIG. 3B: Indicated mice were injected with poly-IC as described (Miyazaki et al., 1996). Freshly isolated DX5⁺ cells were analyzed in a 4 hr ⁵¹Cr release against indicated tumor cell lines. Control mice (open circles), KΔY75/KΔY75 mice (filled circles). In similar experimental conditions, no difference in the lysis of YAC-1 and RMA cell lines was observed, when KΔY75/KΔY75 DX5⁺ cells and control DX5⁺ cells were compared.

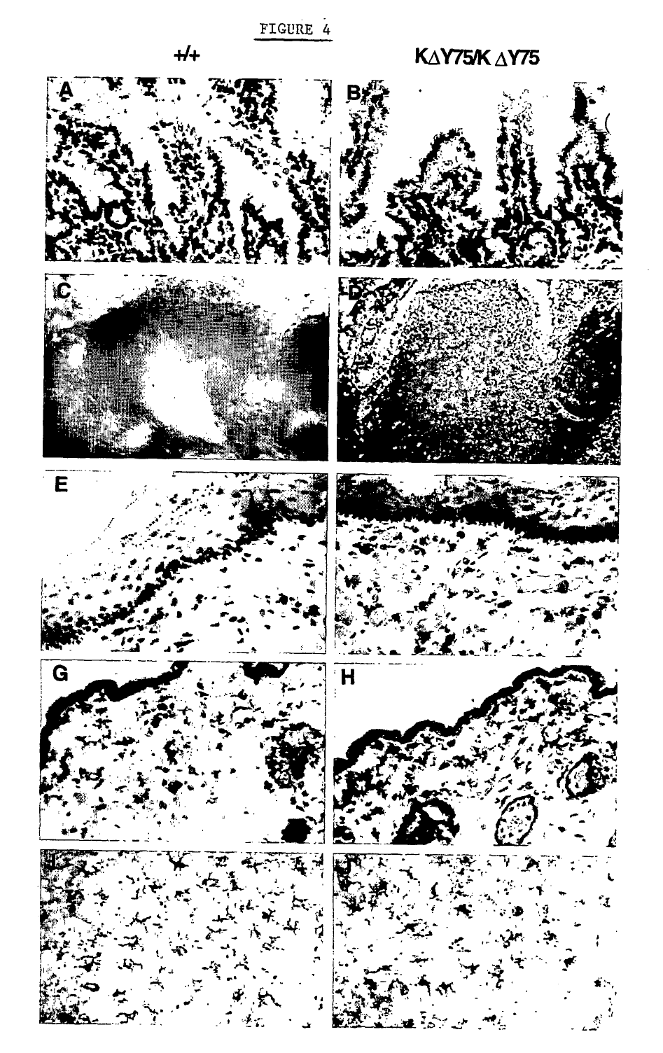

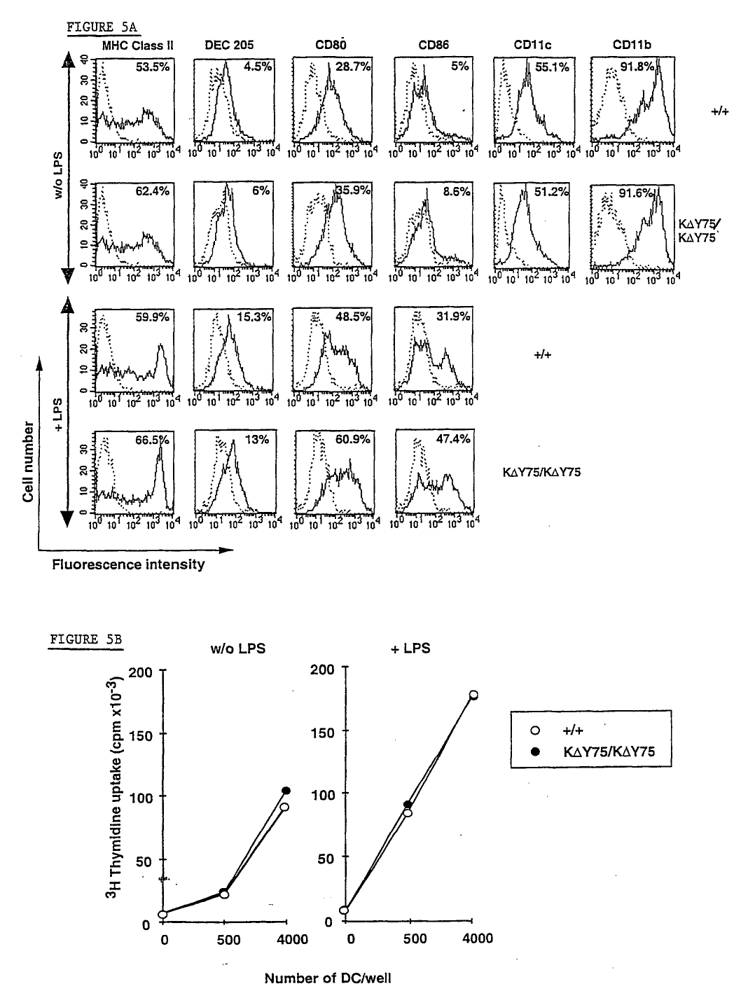

FIGS. 4A-4J : Accumulation of DCs in mucosal tissues and skin from KΔY75/KΔY75 mice.

Immunohistochemical analysis were performed in control mice (left panel:

FIGS. 4A, 4C, 4E, 4G and 4I) and in KΔY75/KΔY75 mice (right panel:

FIGS. 4B, 4D, 4F, 4H and 4J).

FIGS. 4A to 4D: CD11c staining of cryostat sections of small intestine

(FIGS. 4A, 4B) and Peyer's patches (FIGS. 4C, 4D).

FIGS. 4E to 4H: MHC class II staining of sections of buccal mucosa

(FIGS. 4E, 4F) and abdominal skin (FIGS. 4G, 4H).

FIGS. 4I and 4J: DEC205 staining of epidermal sheets.

Counter staining was performed using hematoxylin (final magnification: ×400).

The number and distribution of DCs in skin and mucosa of control mice was comparable in 129, C57BL/6, Balb/C mice and control mice (+/+ and +/KΔY75 littermates), therefore excluding that abnormal accumulation of DCs in KΔY75/KΔY75 mutant mice, merely resulted from a mixed genetic background. The results are representative of data obtained in an average of 3 to 10 tissue sections from individual mice in groups of 3 to 5 mice.

FIGS. 5A and 5B: Phenotypic and functional analysis of BM-DCs from KΔY75/KΔY75 mice.

BM-DCs were differentiated in vitro from bone marrow progenitors in the presence of GM-CSF. On day 6 of culture, LPS (10 ng/ml) was added to some wells and cells were analyzed on day 7.

In FIG. 5A: Phenotypic analysis of untreated (upper panels) or LPS-treated (lower panels) BM-DCs from control mice and KΔY75/KΔY75 mice were carried out by flow cytometry. The percentages of positive stained cells (continuous lines), as well as the control staining using isotype-matched control mAbs (dotted lines) are indicated.

In FIG. 5B: Allostimulatory function of KΔY75/KΔY75 BM-DCs for CD4⁺. T cells was carried out by co-cultivation of untreated or LPS-treated BM-DCs (4×10³/well) from either control (open circles) or KΔY75/KΔY75 (filled circles) mice, with purified allogeneic CD4⁺ T cells (10⁵/well) for 4 days. The results are expressed as mean cpm ±SD of quadruplicate cultures.

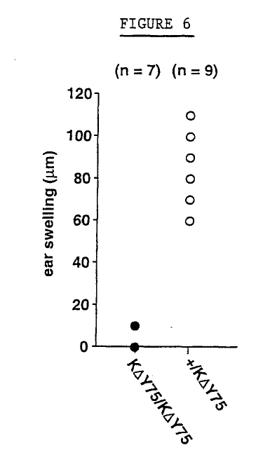

FIG. 6: Impaired CS to DNFB in KΔY75/KΔY75 mice.

Control mice (open circles) and KΔY75/KΔY75 mice (filled circles) were sensitized with 0.5% DNFB and challenged 5 days later onto the right ear with 0.2% DNFB; the left ear received the vehicle alone. CS was determined by the increase in the thickness of the challenged ear (expressed in μM). Medians ±SD are indicated.

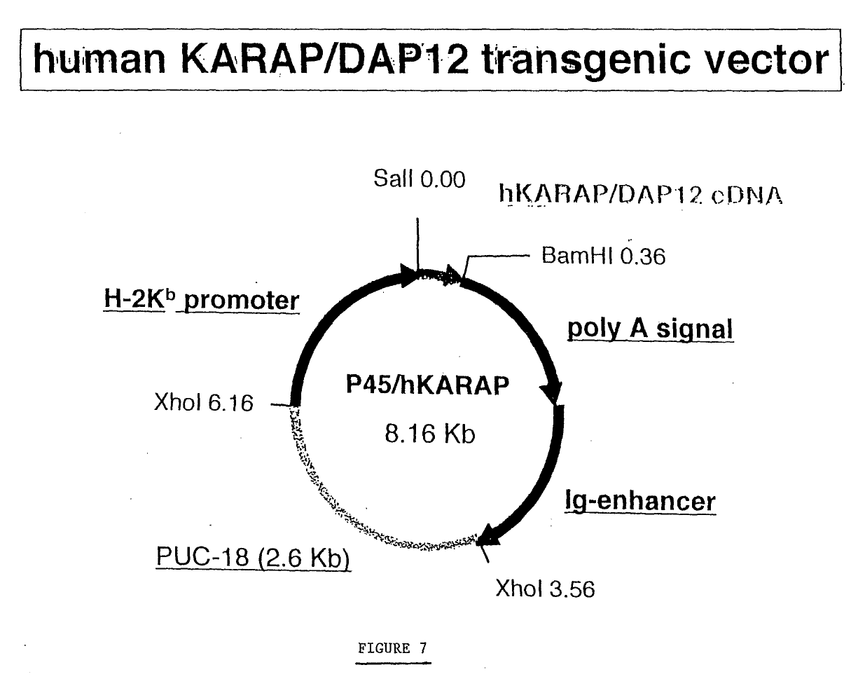

FIG. 7: human KARAP/DAP12 transgenic vector.

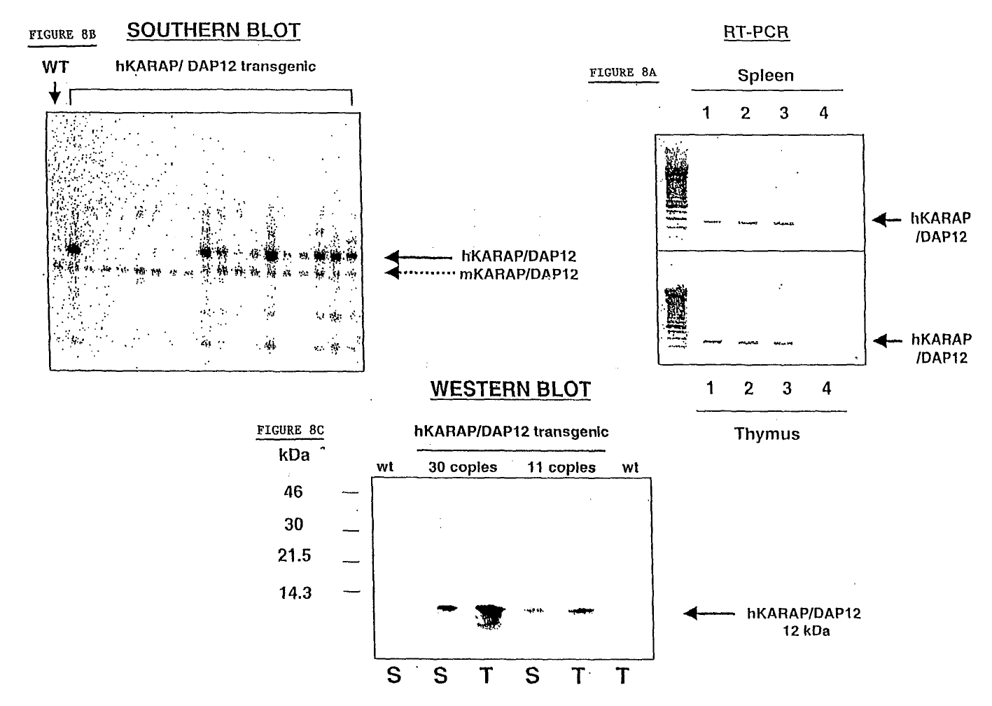

FIGS. 8A, 8B and 8C: transgenic mice contain human KARAP.

In FIG. 8A: RT PCR analysis of spleen and thymus RNA of hKARAP transgenic animals using human KARAP specific oligonucleotides. Lanes 1,2,3 transgenic animals, lane 4 wild type animal.

In FIG. 8B: Southern blot analysis of transgenic mice having integrated copies of human KARAP.

In FIG. 8C: Western blot analysis using anti human KARAP specific, antiserum of spleen and thymus cells in wild type (wt) or transgenic having integrated 11 or 30 copies of human KARAP.

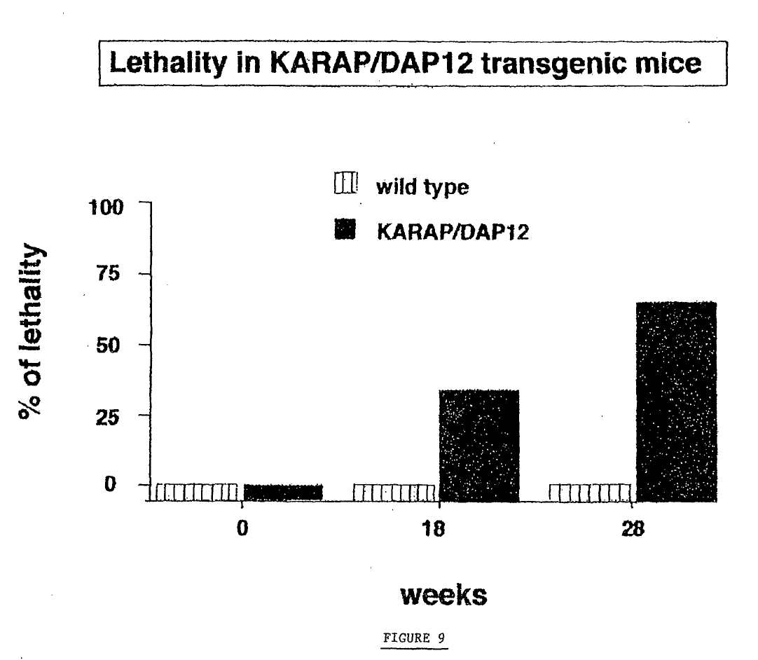

FIG. 9: Lethality in KARAP/DAP 12 transgenic mice having integrated 30 copies of human KARAP.

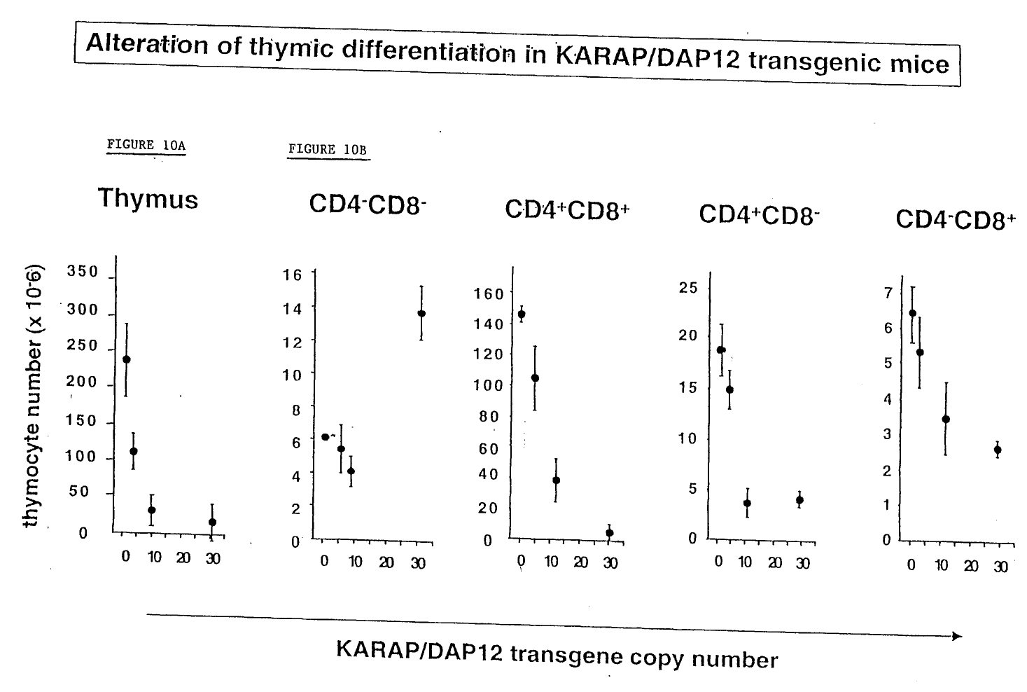

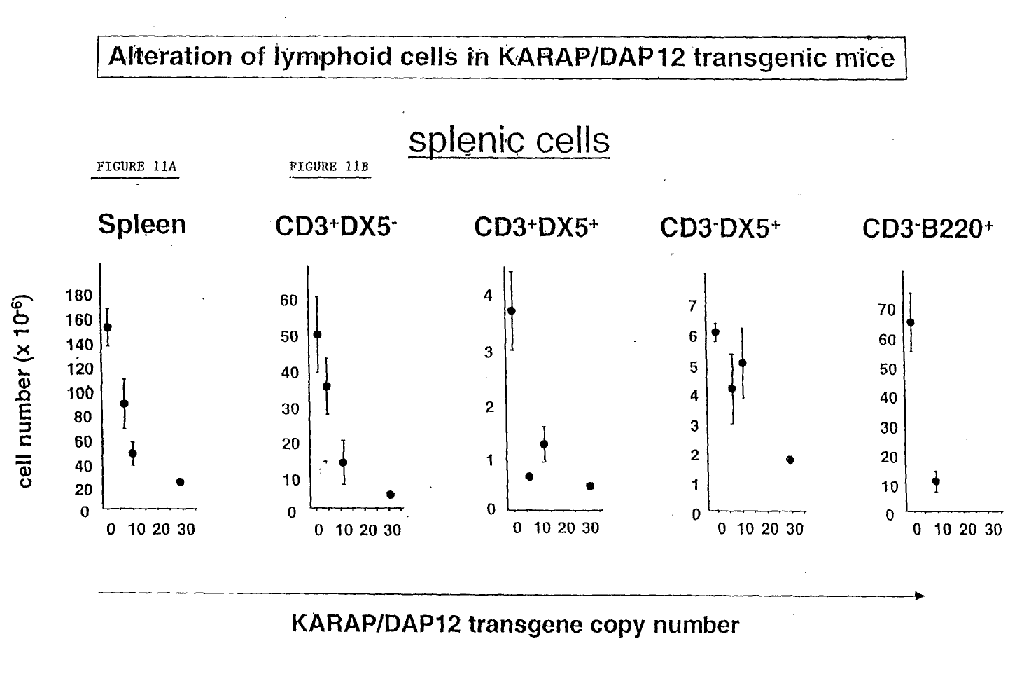

FIGS. 10A and 10B: Alteration of thymic differentiation in KARAP transgenic mice analyzed by flow cytometry.

Cells of the thymus of animal having integrated different number of copies of hKARAP are counted and analyzed by flow cytometry with anti CD4 and anti CD8 antibodies. The total number of thymocytes (FIG. 10A) and cells expressing or not CD4, CD8 (FIG. 10B) are plotted against the number of copies or the transgene. Results are shown as the mean and standard deviation of results obtained from at least 3 animals.

FIGS. 11A and 11B: Alteration of lymphoid compartment in KARAP transgenic mice analyzed by flow cytometry.

Cells of spleen of animal having integrated different number of copies of hKARAP are counted and analyzed by flow cytometry with anti CD3 (T cells), DX5 (NK cells), and B220 (B cells) antibodies. The total number of splenocytes (FIG. 11A) and cells expressing or not CD3, DX5 and B220 (FIG. 11B) are plotted against the number of copies of the transgene. Results are shown as the mean and standard deviation of results obtained from at least 3 animals.

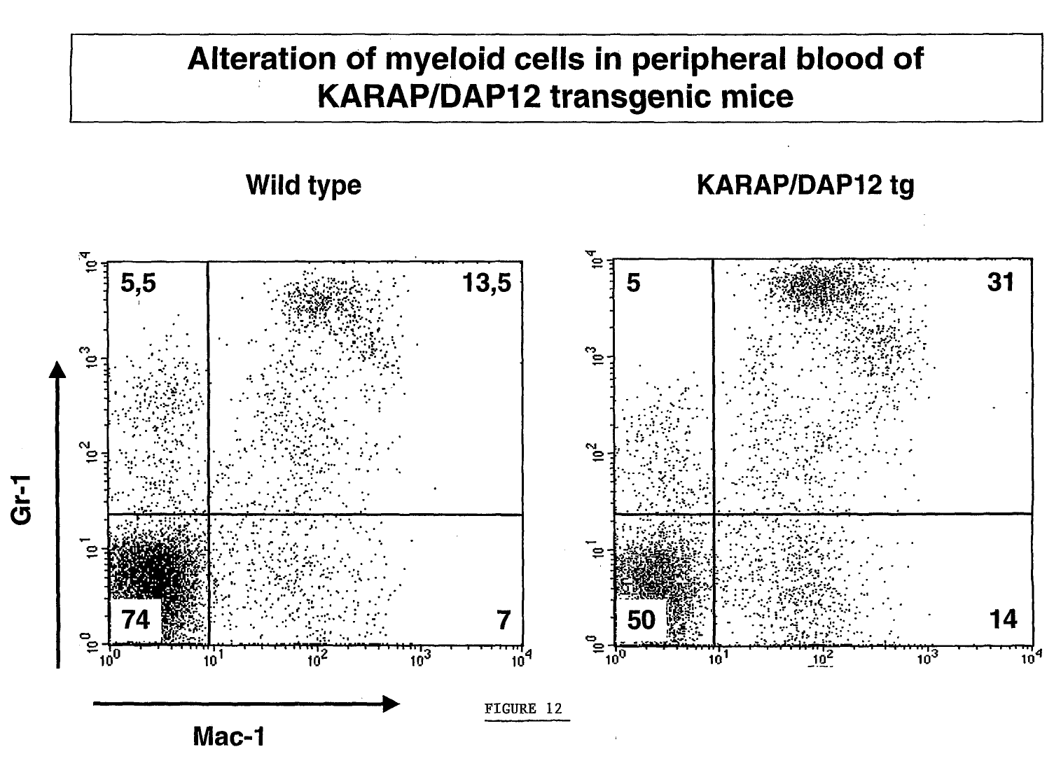

FIG. 12: Augmentation of the myeloid compartment in peripheral blood of KARAP transgenic animals.

Peripheral blood cells of wild type and transgenic animals are stained with Mac-1 and Gr1 antibodies. Percentage of cells negative and positive for the two markers are indicated in the corresponding quadrants, set with irrelevant antibodies. Left panel: wild type mice. Right panel: KARAP/DAP12 mice.

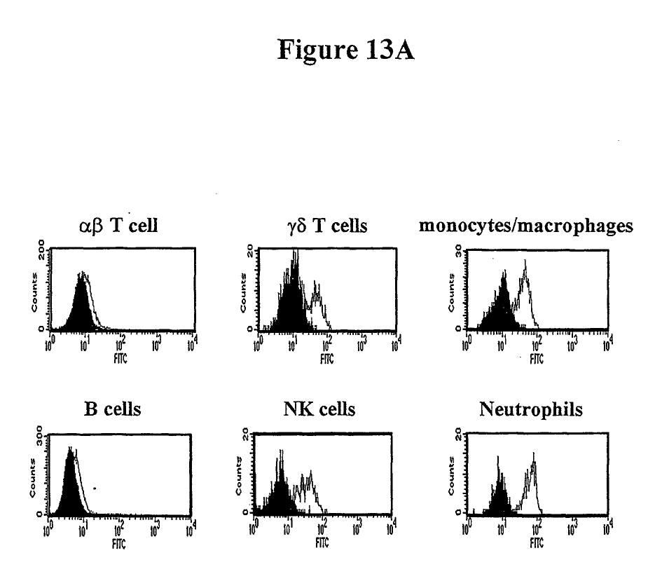

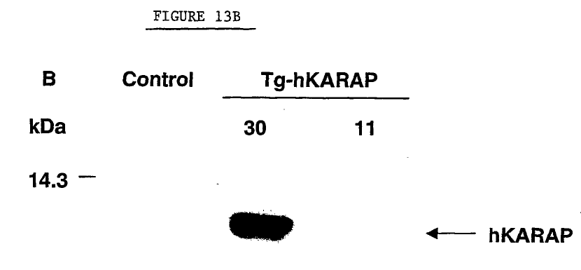

FIG. 13: KARAP/DAP12 expression in control and Tg-hKARAP mice

A) Mouse splenocytes prepared from non transgenic mice were analyzed by three or four color flow cytometry for the intracytoplasmic expression of KARAP/DAP12 using anti-mouse KARAP/DAP12 antiserum (27) on lymphoid and myeloid populations. Histograms were gated on indicated cell subsets as determined by the following cell surface staining: TCRαβ⁺ TCRγδ⁻⁽ αβ T cells), TCRαβ⁻TCRγδ⁺, (γδ T cells), CD3ε⁻CD19⁺ (B cells), CD3ε⁻NK1.1⁺ (NK cells), CD11b⁺ Ly-6G^{-/low} (monocytes/macrophages) and CD11b⁺Ly-6G^{high} (neutrophils). Results are representative of a minimum of 3 independent experiments.

B) Splenic whole cell lysates (50 μg protein/sample) prepared from Tg-hKARAP11, Tg-hKARAP30 mice and non-transgenic littermates (control) were separated on a 12% SDS-PAGE, and immunoblotted using a rabbit anti-human KARAP/DAP12 antiserum.

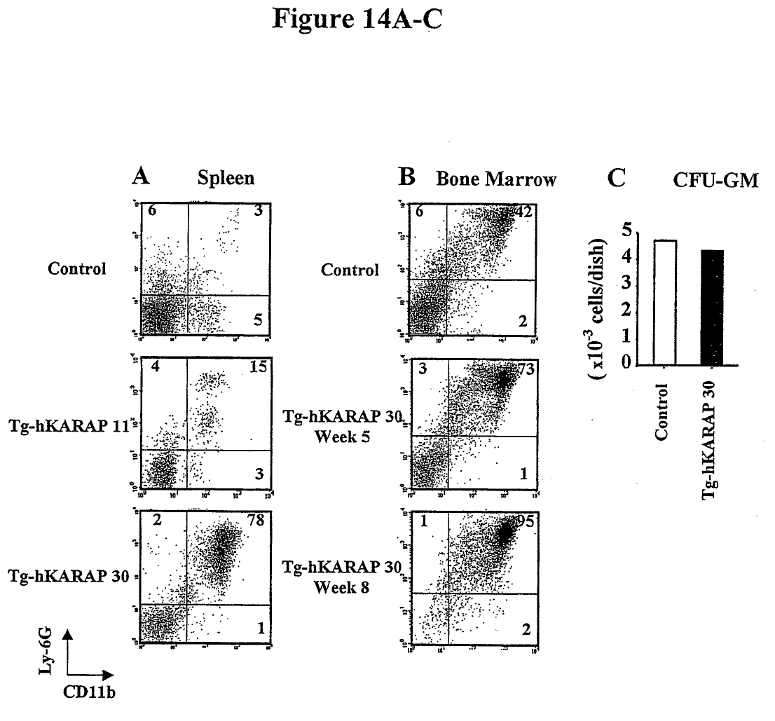

FIG. 14: Neutrophilia in Tg-hKARAP mice

A-B) Cells isolated from Tg-hKARAP11, Tg-hKARAP30 mice and non-transgenic littermates (control) were prepared from the indicated tissues and analyzed by two-color flow cytometry for the cell surface expression of CD11b and Ly-6G. The frequencies of each myeloid sub-population are indicated in their respective quadrants; results are representative of a minimum of 3 independent experiments.

C) Bone marrow cells were cultured in the presence of 5 μg/ml of recombinant mouse Granulocyte Macrophage Colony Stimulating Factor (GM-CSF ; R&D system, Inc) in 1% methylcellulose containing medium. After 12 days, colonies were scored and mixed prior to cell counting and flow cytometry analysis using anti-CD3ε+anti-B220 mAbs or anti-CD11b +anti-Ly-6G mAbs. Results of one representative out of 3 independent experiments were expressed as the absolute number of CFU-GM cells (CD11b⁺Ly-6G⁺) per bone marrow cultures.

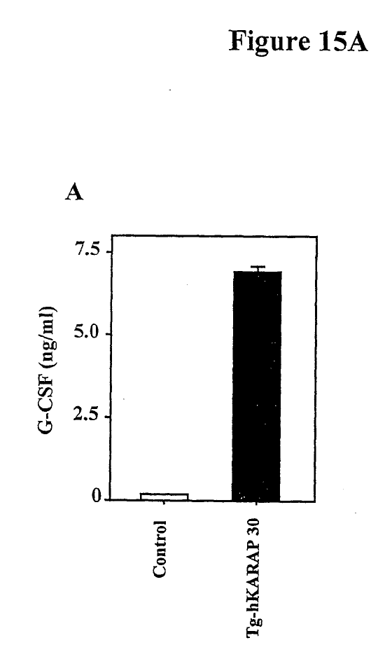

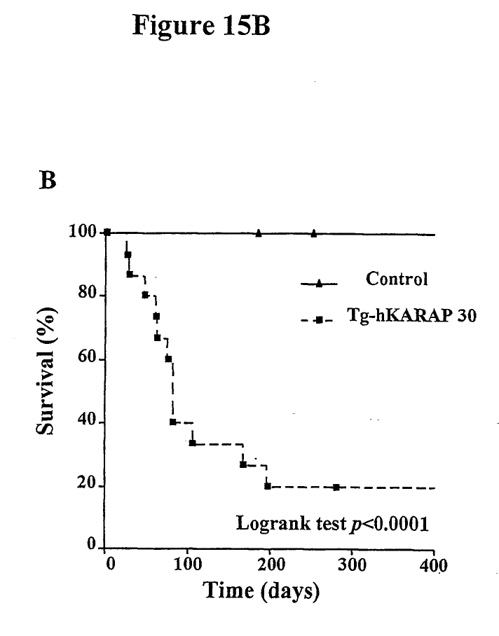

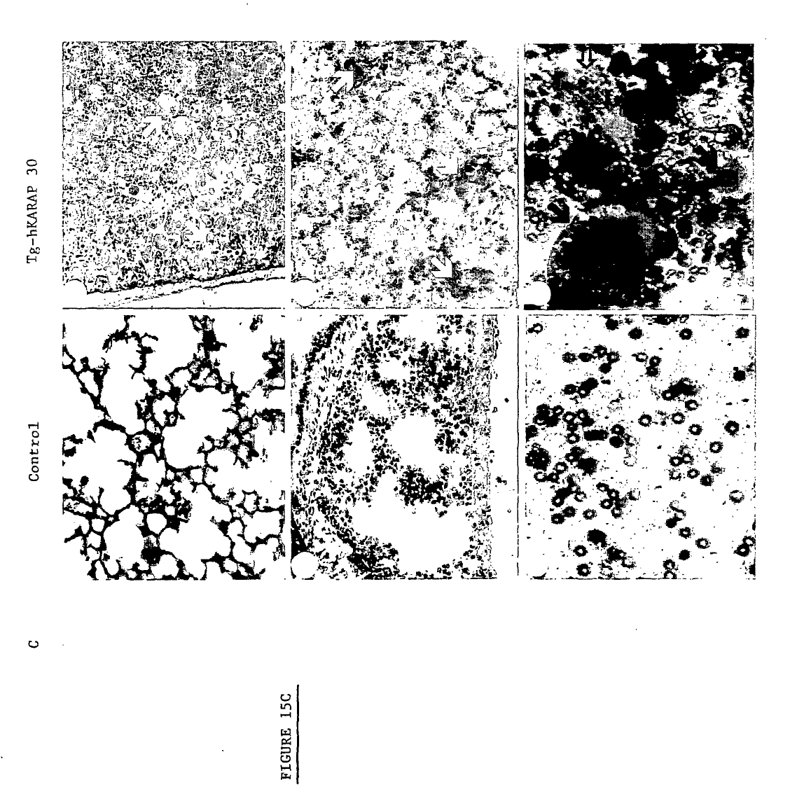

FIG. 15: Fatal inflammatory syndrome in Tg-hKARAP mice

A) Serum levels of G-CSF were measured using ELISA. Results are expressed as the mean ±SD of G-CSF concentration in serum in groups of non-transgenic littermates (n=4) and sick Tg-hKARAP30 mice (n=3). Asterisk indicates statistically significant difference between two groups (*P<0.05).

B) Mix gender cohorts of Tg-hKARAP30 mice (n=12) and non-transgenic littermates (control) (n=14) were set aside after successful weaning and studied for 400 days. Survival is plotted according to the method of Kaplan and Meier, and P values are based on comparisons using the log-rank test (P<0.0001). All data were computed using SPSS for Windows software.

C) Immunohistochemical analysis was performed as followed: organs were snap-frozen in liquid nitrogen and stored at −80° C. until use, and colorations were performed on 5 μm-thick serial cryostat sections (Cryostat Reichert-Jung, 2800 R, Leica). Upper panels: Hematoxylin-Phloxin-Safran histopathology of representative lung sections prepared from control littermates (left) and sick Tg-hKARAP30 mice (right). Intra-alveolar multinucleated invasive macrophagic cells are indicated ( [symbol] ). Middle panel: Polyclonal rabbit anti-human KARAP/DAP12 was used at the 1/50 dilution (3), and the sections were incubated for 30 min at room temperature with biotinylated anti-rabbit antibodies and then visualized by avian-biotin peroxidase (Kit vectastain, Vector). Lower panels: May-Grünwald-Giemsa staining of lung impressions from control littermates (left) and sick Tg-hKARAP30 mice (right) with multinucleated invasive cells ( [symbol] ) and platelet accumulation ( [symbol] ). Data are from one representative experiment of three that yielded similar results.

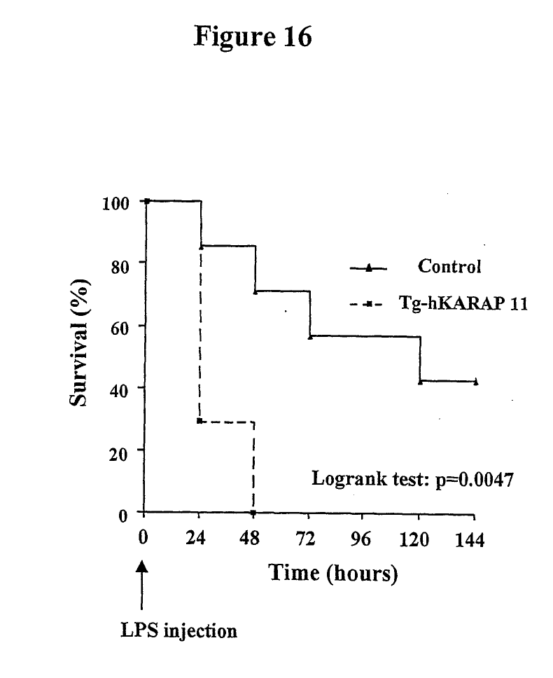

FIG. 16: Increased LPS-sensitivity of Tg-hKARAP mice.

Tg-hKARAP11 mice (n=7), non-transgenic littermates (control) (n=6) (8-10 weeks) were injected i.p. with 700 μg LPS from E. coli 055:B5 (Sigma). Survival is plotted according to the method of Kaplan and Meier, and P values are based on comparisons using the log-rank test (P=0.0047).

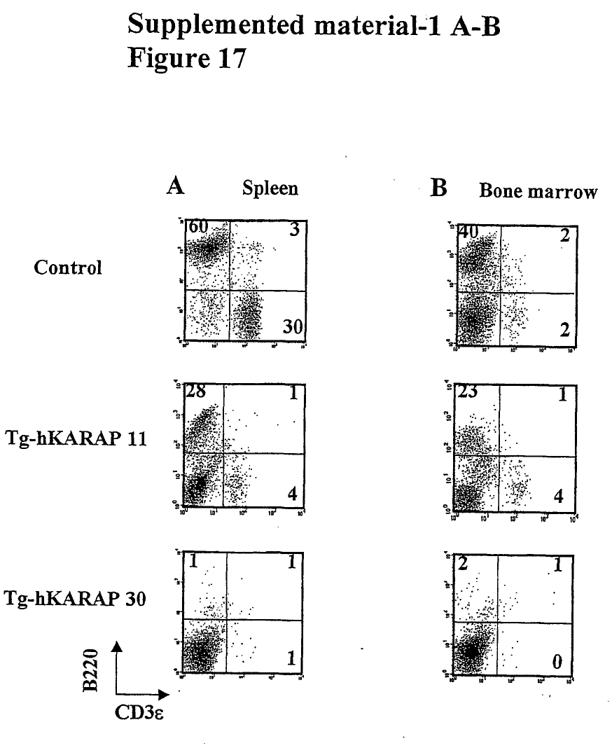

FIG. 17: supplemented material-1 A-B.

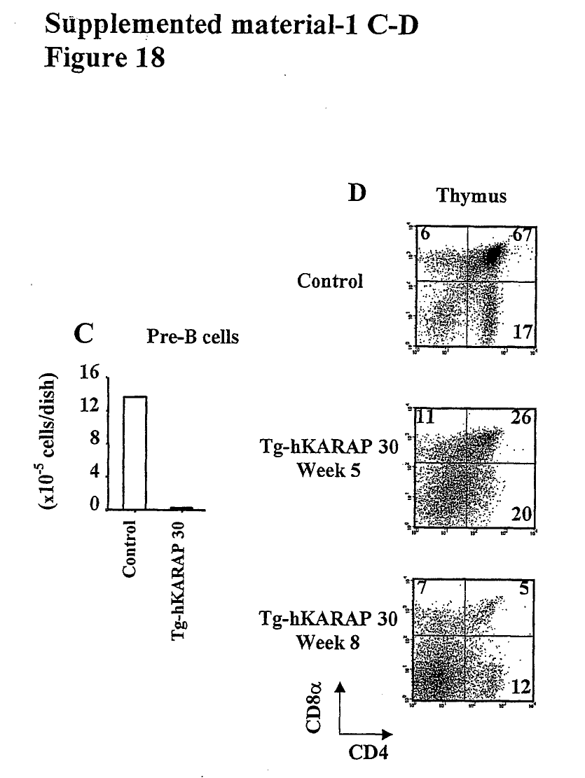

FIG. 18: supplemented material 1 C-D.

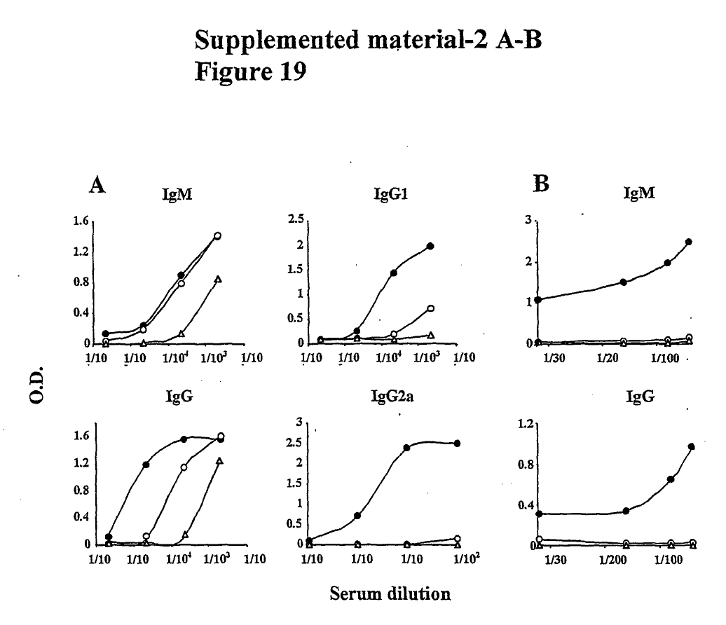

FIG. 19: supplemented material-2 A-B.

### EXAMPLE 1

Combined Natural Killer Cell and Dendritic Cell Functional Deficiency in KARAP/DAP12 Loss-of-Function Mutant Mice

KARAP/DAP12 is a transmembrane polypeptide with an intracytoplasmic Immunoreceptor Tyrosine-based Activation Motif (ITAM). KARAP/DAP12 is associated with several activating cell surface receptors in hematopoietic cells. Here we report that knock-in mice bearing a non-functional KARAP/DAP12 ITAM present altered innate immune responses. Although in these mice NK cells are present and their repertoire of inhibitory MHC class I receptors is intact, the NK cell spectrum of natural cytotoxicity towards tumor cell targets is restricted. KARAP/DAP12 loss-of-function mutant mice also exhibit a dramatic accumulation of dendritic cells in muco-cutaneous epithelia, associated with an impaired hapten-specific contact sensitivity.

Thus, despite its homology with CD3ζ and FcRγ, KARAP/DAP12 plays a unique role in innate immunity, emphasizing the non-redundancy of these ITAM-bearing polypeptides in hematopoietic cells.

Introduction

The consensus intracytoplasmic ITAM sequence YxxL/Ix₆₋₈YxxL/I has led to the identification of a group of ITAM-bearing transduction polypeptides which are associated with multiple cell surface receptors. Single charged amino-acid residues in the transmembrane domains of both ITAM-bearing polypeptides and their associated receptors are critical for the formation of these functional oligomeric complexes. The group of ITAM-bearing polypeptides includes CD3 molecules (CD3γ, CD3ζ, CD3ε) associated with the T cell receptor complex (TCR), Ig-α and Ig-β molecules associated with the B cell receptor complex (BCR), as well as CD3ζ and the closely related FcRγ, which associate with some TCR and FcRs.

KARAP/DAP12 is an ITAM-bearing disulfide-linked dimer, closely related to CD3ζ and FcRγ, which associates with a variety of cell surface receptors on NK cells and on myeloid cells (WO 98/49292). In NK cells, KARAP/DAP12 associates with the activating isoforms of inhibitory receptors for classical MHC class Ia molecules, i.e. activating Killer cell Ig-like Receptors (KIR-S; previously referred to as KAR) in humans as well as Ly49D and Ly49P in the mouse (WO 98/49292). In humans and mice, KARAP/DAP12 also associates with the CD94/NKG2C activating receptors for the HLA-E and Qa-1 MHC class Ib molecules respectively. In humans, KARAP/DAP12 associates with NKp44, a NK cell surface receptor involved in triggering NK cell activation programs. In monocytes, KARAP/DAP12 dimers associates with the lectin-like MDL-1 molecules, as well as the Ig-like Signal Regulatory Protein β1 (SIRP-β1), TREM-1 and TREM-2 receptors, whose ligands and function are still unveiled.

ITAM-bearing polypeptides couple cell surface receptors to signaling pathways which depend on protein tyrosine kinases (PTKs), and integrity of the ITAM sequence is mandatory for the signaling function of these transduction polypeptides. To dissect the role of oligomeric complexes that associate with KARAP/DAP12, we generated KARAP/DAP12 loss-of-function mutant mice (KΔY75/KΔY75), in which the KARAP/DAP12 ITAM is non-functional. We describe here the alterations of NK and dendritic cell (DC) subsets observed in KΔY75/KΔY75 mice.

Experimental Procedures

Mice

C57BL6, Balb/c, 129 and CBA mice were from Jackson laboratories. C57 black 6 mice transgenic for Cre recombinase gene controlled by a CMV promoter has been produced by standard techniques and has been described previously (Chwenk F, Baron U, Rajewsky K. Nucleic Acids Res. December 1995 25;23(24):5080).

C57BL6 CD3ζ-FcRγ^{-/-} double knockout mice have been previously described (Shores et al. 1998, J. Exp. Med. 187:1093-1101 of which content is herewith incorporated by reference). All mice were reared under specific-pathogen free animal facilities. Control mice include +/+ and +/KΔY75 littermates, for which no significant phenotypic and functional differences were detected.

Generation of KΔY75/KΔY75 Mice

An EcoRI/XhoI-digested genomic fragment of 8.8 kb containing mouse KARAP/DAP12 gene (WO 98/49292 of which content is herewith incorporated by reference), was sub-cloned in pBlueScript KS plasmid. A neo-resistance cassette flanked by two lox P sites was inserted at the unique XcaI restriction site. The first loxp sequence is 5′ of the KARAP/DAP12 sequence corresponding to the second YxxL within KARAP/DAP12 ITAM. This insertion generated the mutant KARAP/DAP12 gene (KΔY75) encoding a KARAP/DAP12 protein in which the wild type C-terminal Y₇₅-R₈₆ amino-acid stretch (YSDLNTQRQYR) is replaced by a G₇₅-I₉₀ peptide (GLQEFIEDEKKKRNSI), with no homology with any peptide sequences in the databases. 129 Ola ES (E14) cells were transfected by electroporation with the linearized targeting construction and ES clones were selected by Southern blot. Recombinant progeny of chimeric mice was mated with C57BL/6 Cre transgenic mice. +/KΔY75 heterozygous neo-excised progeny was back-crossed to obtained homozygous KΔY75/KΔY75 mutant mice. Sequence analysis of RT-PCR products obtained from KΔY75/KΔY75 splenocytes confirmed the replacement of the wild type KARAP/DAP12 Y₇₅-R₈₆ C-terminus by the predicted G₇₅-I₉₀ peptide.

Southern Blot and RT-PCR Analysis

Genomic DNA extracted from tails of KΔY75/KΔY75 mice was cut with EcoRI enzyme and analyzed by Southern Blot using radiolabelled 0.4 kb XhoI/EcoRI fragment as probe E. Total RNA was extracted from splenocytes of KΔY75/KΔY75 mice and converted in cDNA as previously described (WO 98/49292). cDNA was then tested by RT-PCR using the following primers:

β-actin forward (5′-TACCACTGGCATCGTGATGGACT-3′);
β-actin reverse (5′-TCCTTCTGCATCCTGCGGCAAT-3′);
KARAP/DAP12 forward (5′-ACTTTCCCAAGATGCGAC-3′);
KARAP/DAP12 reverse (5′-GTACCCTGTGGATCTGTA-3′);
DAP10 forward (5′-ATGGACCCCCCAGGCTACCTC-3′);
DAP10 reverse (5′-TCAGCCTCTGCCAGGCATGTT-3′).

Cells

YAC-1 (ATCC no. TIB-160), EL4 (ATCC no. TIB-39), RMA, RMA-S (Townsend et al. 1989 Nature vol. 340: 443-448), J774 (European animal cell culture collection n^{o}85011428) cell lines were used.

All cell lines were cultured in RPMI 1640 10% FCS with sodium pyruvate (1 mM) and β-mercaptoethanol (5×10⁻⁵ M).

IL-2-activated killer cells were obtained as follows: freshly isolated splenocytes were incubated with MACS DX5-coupled beads (Miltenyi) and then subjected to positive selection. Collected DX5⁺ cells were cultured for 7 days in RPMI 10% FCS with sodium pyruvate (1 mM), β-mercaptoethanol (5×10⁻⁵ M), non-essential MEM amino-acids, Hepes (10 mM) and 1000 UI/ml of IL-2 (Chiron).

Antibodies

The following mAbs were obtained from Pharmingen-Becton Dickinson: APC-conjugated anti-CD3ε (2C11, hamster IgG1), PE-conjugated DX5 (DX5, Rat IgM), FITC-conjugated anti-Ly49D (4E5, rat IgG2a), FITC-conjugated anti-Ly49C/I (5E6, mouse IgG2a), FITC-conjugated anti-Ly49G2 (4D11, rat IgG2a).

Purified anti-Ly49D (4E5, rat IgG2a) and anti-NKG2A/C (20D5, rat IgG2a) mAbs have been previously described (Mason et al. 1998 J. Immunol. 160:4148-4151; Vance et al. 1999 J. Exp. Med. 190, 1801-1812 of which contents are herewith incorporated by reference). Specific anti-Ly49A mAbs (mouse IgG1) can be obtained from Pharmingen.

Isotype-matched control mAbs were purchased from Pharmingen.

Cytotoxicity Assay

8-12 weeks old mice were injected i.p. with 150 μg/mouse of polyinosinic:cytidylic acid (poly I:C, Sigma) 8 to 48 hr prior to sacrifice, and freshly isolated splenocytes were used as effector cells. Alternatively, IL-2activated DX5⁺ purified splenocytes were tested after 7 days of culture in IL-2. Effector cells were used in a standard 4 hr ⁵¹Cr cytotoxicity assay (Olcese et al., 1997 J. Immunol. 158: 5083-5086 of which content is herewith incorporated by reference). For Ly49D-redirected killing, IL-2-activated cells were tested against the tumor cell line P815 in the presence of 5 μg/well of purified anti-Ly49D mAbs.

Immunohistochemical Analysis

Cryostat section (5 μm) of various tissues were acetone-fixed and stained using hamster anti-mouse CD11c mAb (N418, ATCC) as neat supernatant, rat anti-mouse mannose receptor mAb DEC205 (NLDC-145, Biotest, France) as neat supernatant, rat anti-mouse MHC class II mAb CD311 reacting with I-A and I-E molecules in all mouse haplotypes, as 1/10 dilution of supernatant. Biotin-conjugated anti-CD11b mAb (M1/70) specific for Mac-1 α-chain (1/100 dilution) and control isotypes including hamster IgG were from Pharmingen or ATCC. Endogenous peroxidase was blocked by treating tissue sections with 0.3% H₂O₂, prior to incubation with the primary antibodies. In other experiments, epidermal sheets from ear skin were incubated overnight at 4° C. with specific or control isotype matched mAb. Specific staining of cryostat sections and epidermal sheets was revealed using biotinylated mouse adsorbed F(ab′)₂ fragment of goat anti-rat IgG (Vector, Biosys, France) or goat F(ab′)₂ anti-hamster IgG (H+L) (Pierbio Science, France), followed by streptavidin conjugated to peroxidase (Amersham, France). The reaction was developed using AEC substrate and H₂O₂ (AEC kit, Dako), and the tissue sections were counter-stained with hematoxylin (Dako). DC were counted from 3 to 10 tissue sections of individual mice using a microscopic grid and the results expressed as number of DCs/mm2 of tissue (skin and buccal mucosa) or number of DCs/intestinal villus.

BM-DCs

BM-DCs were generated from bone marrow progenitors: bone marrow was flushed from tibias and femurs prior to red blood cell depletion using 0.83% ammonium chloride. Cells were cultured at 37° C. in 24-well culture plates (2.105 cells/ml/well) in complete RPMI medium supplemented with 40 ng/ml of recombinant murine GM-CSF (Peprotech, France). Half of the medium was replaced every other day by fresh medium and GM-CSF. On day 6 of culture, BM-DCs were stimulated for 24 hr at 37° C. with E. Coli LPS (10 ng/ml, Sigma, La Verpilliere, France). Loosely attached BM-DCs were harvested on day 7, washed twice in PBS containing 1% BSA and 0.01% sodium azide and then stained with the following antibodies: FITC-conjugated hamster anti-mouse CD11c mAb (HL-3), FITC- or biotin-conjugated CD11b mAb (M1/70), PE-conjugated MHC class II mAb (M5/114, a rat mAb anti-IA^{b/d/q}, I-E^{d/k}), FITC-conjugated CD86 mAb (GL-1), FITC-conjugated hamster anti-mouse CD80 (16-10A1), all from Pharmingen. DEC205 (NLDC-145) was used as culture supernatant from Biotest. Control isotypes included FITC-conjugated hamster IgG and PE- or FITC-conjugated rat IgG2aK (Pharmingen). DEC205 staining was revealed using mouse-adsorbed FITC-conjugated F(ab′)₂ goat antibody specific for rat IgG (H+L) (Caltag Laboratories). Before staining, Fc receptors (FcγRII/III) were blocked using either 5% normal mouse serum or rat anti-mouse CD16/CD32 (2.4.G2) for 30 min on ice. Samples were analyzed using a FACStar and Lysis II software (both from Becton Dickinson).

Allogeneic Mixed Lymphocyte Reaction

Untreated and LPS-treated day 7 BM-DCs were treated with 25 μg/ml of mitomycin C and co-cultured in quadruplicate wells of round-bottomed microculture plates with 10⁵ CD4⁺ T cells purified from CBA mice spleen using MACS anti-CD4-coupled microbeads (Miltenyi, France). On day 3 of co-culture, 1 μCi [³H] thymidine (specific activity 1 Ci/mM) was added to each well and T cell proliferation was determined by tritiated thymidine incorporation during the last 24 hr of culture. The cultures were harvested with an automatic cell harvester and radioactivity was counted using a B plate counter (Wallac). The results are expressed as mean cpm ±SD in quadruplicate wells.

Contact Sensitivity

CS to DNFB was determined by the Mouse Ear Swelling Test (MEST), as previously described (e.g. Desvignes et al. 1998 Clin. Exp. Immunol. 113: 386-393 of which content is herewith incorporated by reference).

Briefly, mice were sensitized epicutaneously on day 0 by application of 0.5% DNFB (25 μl) diluted in acetone/olive oil (4/1, v/v) onto 2 cm² of shaved abdominal skin. On day 5 after sensitization, mice were challenged onto the right ear by topical application of 0.2% DNFB, whereas the left ear received the vehicle alone (acetone/olive oil). Contact sensitivity was determined by increase in the thickness of the challenged ear compared with that of control left ear and was expressed in μm: (T-To of the right ear)—(T—To of the left ear), where T and To represent the values of ear thickness after and before challenge, respectively.

Results

Generation of KARAP/DAP12 Knock-in Mice

In vitro studies using mutant KARAP/DAP12 have shown that both Y residues in ITAMs are necessary for KARAP/DAP12 signaling function. We thus designed a KARAP/DAP-12 knock-in strategy in which the mutated KARAP/DAP12 protein lacks the Y75 residue and wild-type C-terminus amino-acids (KΔY75 protein). A homologous recombination targeting vector was constructed by inserting a neo-resistance cassette flanked by two lox P sites at the unique XcaI restriction site (FIG. 1A). In order to obtain KΔY75/KΔY75 mice, ES cells were transfected with the targeting construction (FIG. 1B) and recombinant ES clones were injected in Balb/c blastocystis. Recombinant offspring of chimeric mice was crossed with Cre mice transgenic, generating “floxed” +/KΔY75 mice (FIG. 1B). Heterozygous +/KΔY75 mice were mated to obtain KΔY75/KΔY75 mice, and offspring was analyzed by Southern Blot (FIG. 1C). To confirm the replacement of wild type KARAP/DAP12 by KΔY75, RT-PCR amplification was performed on total splenocytes RNA prepared from +/+, +/KΔY75 and KΔY75/KΔY75 mice. As shown in FIG. 1D, amplification of wild type KARAP/DAP12 cDNA was absent in KΔY75/KΔY75 mice and substituted with a band of larger size (due to the presence of one loxp site). The DAP-10 transduction protein is encoded by a gene located at ˜100 bp of the KARAP/DAP12 gene in a tail-to-tail orientation (Chang et al. 1999 J. Immunol. 163: 4651-4654; Wu et al. 1999 Science 285: 730-732 of which contents are herewith incorporated by reference). RT-PCR analysis of DAP-10 expression was similar in +/+, +/KΔY75 and KΔY75/KΔY75 mice (FIG. 1D), indicating that the integrity of genes flanking the targeted KARAP/DAP]2 gene was preserved in KΔY75/KΔY75 mice. KΔY75/KΔY75 mice were obtained at Mendelian frequencies, developed normally and were fertile. In addition, no statistically significant variations in the numbers of lymphoid and myeloid subsets were detected using immunofluorescent flow cytometry analysis when peripheral blood mononuclear cells (PBMCs) from +/+, +/KΔY75 and KΔY75/KΔY75 mice were compared.

NK Cell Repertoire of Ly49 Molecules in the Presence of Non-Functional KARAP/DAP12 Molecules

In mouse NK cells, association with KARAP/DAP12 has been shown to be required for the cell surface expression of Ly49D. No significant alteration in the reactivity of anti-Ly49D mAb (4E5) was observed when splenic NK cells derived from KΔY75/KΔY75 mice and from control mice were compared (FIG. 2A), consistent with the cell surface association of KΔY75 with Ly49D in KΔY75/KΔY75 mice. However, anti-Ly49D mAbs were unable to trigger the lysis of the FcR⁺ tumor target cells P815 by KΔY75/KΔY75 NK cells, in contrast to results obtained with control NK cells (FIG. 2B, left panel). Ly49D has been recently shown to be involved in the cytotoxicity exerted by mouse NK cells against the xenogeneic target cells CHO (Idris et al., 1999 Proc. Natl. Sci. USA 96: 6330-6335 of which content is herewith incorporated by reference). Consistent with this result, the ability of NK cells derived from KΔY75/KΔY75 mice to induce the lysis of CHO cells was severely impaired as compared to that of control NK cells (FIG. 2B, right panel). However, the residual lysis of CHO cells by KΔY75/KΔY75 NK cells indicates that receptors distinct from Ly49D might also be involved in this xenogeneic recognition. Nevertheless, the signaling properties of Ly49D are abolished in NK cells from KΔY75/KΔY75 mice, confirming that KARAP/DAP-12 ITAM is required for Ly49D activating properties.

The mechanisms which govern the shaping of the NK cell repertoire of Ly49 molecules are unclear. It has been proposed that activating NK receptors might contribute to the regulation of inhibitory NK receptor expression on NK cells. We thus analyzed in KΔY75/KΔY75 mice the expression of Ly49A, C/I and G2 as well as NKG2A/NKG2C using available mAbs. As shown in FIG. 2A, no significant difference in the cell surface expression of these receptors was observed when splenic NK cells isolated from control and KΔY75/KΔY75 mice were compared. Similar results were obtained when splenic NK cells were cultured in vitro with IL-2. Thus, no alteration of NK cell differentiation can be documented in KARAP/DAP12 loss-of-function mutant mice, in which activating NK receptors for MHC class I molecules are not functional.

Restricted Natural Cytotoxicity Function in KARAP/DAP12 Knock-In Mice

Natural cytotoxicity involves multiple Natural Cytotoxicity Receptors (NCRs) expressed at the surface of NK cells. In humans, NCRs include Ig-like cell surface receptors which are associated with ITAM-bearing polypeptides: NKp46 and NKp30 are associated with CD3ζ and/or FcRγ, whereas NKp44 is, associated with KARAP/DAP12. Control, KΔY75/KΔY75 and mice genetically deficient for both CD3ζ and FcRγ genes (CD3ζ-FcRγ^{-/-} mice) (Shores et al., 1998 cf. supra) were thus used as a source of NK cells to investigate the relative contribution of KARAP/DAP12 and CD3ζ/FcRγ in NK cell natural cytotoxicity. No significant alteration of natural cytotoxicity against YAC-1 was observed when total splenocytes freshly isolated from control and KΔY75/KΔY75 mice were compared (FIG. 3A). The NK cell natural cytotoxicity function exerted against RMA and its MHC class I⁻ variant RMA/S was also similar between control and KΔY75/KΔY75 mice (FIG. 3A). By contrast, the natural cytotoxicity function exerted by freshly isolated KΔY75/KΔY75 DX5⁺ splenocytes against the macrophagic cell lines J774 was severely diminished as compared to that observed with control DX5⁺ cells (FIG. 3B). These data thus indicate that KARAP/DAP12 is involved in NK cell natural cytotoxicity, depending on the nature of the target cell lines. These results also confirm that natural cytotoxicity involves multiple NK cell surface receptors, which are engaged or not, depending on the cell surface expression of their ligands on target cells. Along this line, severe natural cytotoxicity defects against YAC-1, RMA, RMA/S (FIG. 3A) were observed when total splenocytes freshly isolated from CD3ζ-FcRγ^{-/-} mice were used as a source of NK cells. The natural cytotoxicity function exerted by NK cells derived from CD3ζ^{-/-} and FcRγ mice against YAC-1 cells has been reported to remain intact (Liu et al. 1993 EMBO J. 12: 4863-4875; Takai et al., 1994 Cell 76: 519-529). Our data show that NK cells from CD3ζ-FcRγ^{-/-} mice present a major deficiency in natural cytotoxicity towards multiple tumor cell lines. Therefore, CD3ζ and FcRγ appear to exert redundant function in natural cytotoxicity, contrasting with the mandatory function played by FcRγ in ADCC (Takai et al. 1994 cf. supra). Taken together, these results indicate that CD3ζ-FcRγ and KARAP/DAP12 do not exert redundant function in NK cells, but are associated with distinct NCRs which are selectively involved in the natural cytotoxicity towards cognate target cell lines.

Accumulation of DCs in Mucosal Tissues and Skin in KARAP/DAP12 Knock-In Mice

The expression of KARAP/DAP-12 in myeloid cells including DCs (WO 98/49292; Bakker et al. 1999 Proc. Natl. Acad. Sci. USA 96: 9792-9796; Bouchon et al. 2000 J. Immunol 164: 49914995; Dietrich et al. 2000 J. Immunol. 164: 9-12; Tomasello et al. 2000 Eur J. Immunol 30: 2147-2156 of which contents are herewith incorporated by reference) prompted us to investigate whether the absence of a functional KARAP/DAP12 molecule affected the distribution of DCs present in lympho-epithelial tissues. Immunohistochemical staining of DCs on cryostat sections of the small intestine was carried out using anti-CD11c mAb (N418), which recognizes both myeloid CD11b⁺ DCs and lymphoid CD8α⁺ DCs (Iwasaki and Kelsall, 2000 J. Exp. Med. 191:1381-), and anti-DEC205 mAb (NLDC-145), which reacts with interdigitating DCs from the T cell zone of lymphoid organs, including Peyer's patches (Kelsall and Strober, 1996 J. Exp. Med. 183: 237 247). In control mice, Peyer's patch CD11c⁺ DCs formed a layer of cells just beneath the epithelium, i.e. in the subepithelial dome (SED) region, were present in the interfollicular T cell region (IFR) and were also detected as scattered cells in the follicle (FIG. 4C). A limited number of CD11c⁺ DCs were also present in the lamina propria of the small intestinal villi (FIG. 4A). KΔY75/KΔY75 mice exhibited a two-fold accumulation of CD11c⁺ DCs in the intestinal lamina propria (FIG. 4B): 2±0.8 DCs/villus vs. 4±0.6 DCs/villus for control and KΔY75/KΔY75 mice respectively (n=4). A dramatic increase in CD11c⁺ DCs in the SED of Peyer's patches, but no detectable changes in interfollicular CD11c⁺ DCs were observed when control and KΔY75/KΔY75 mice were compared (FIG. 4D). Both control and mutant mice presented comparable numbers of DEC205⁺ DCs, which were found primarily in the IFR but not in the SED. Thus, KΔY75 mutation was associated with increased numbers of CD11c⁺ DEC205⁻ DCs in small intestinal mucosa and SED of Peyer's patches.

MHC class II⁺ DCs, localized in the suprabasal layer of the pluristratified epithelium as well as in the underlying dermis, represent the major antigen-presenting cell (APC) population of the mouse buccal mucosa. Since buccal epithelial DCs, similarly to epidermal Langerhans cells, express high levels of MHC class II molecules but are weakly positive for DEC205 and CD11c, DCs were identified in sections of both buccal mucosa and abdominal skin by staining for MHC class II and by their typical dendritic morphology. A dramatic accumulation of MHC class II⁺ cells with dendritic morphology was observed in the buccal mucosa of KΔY75/KΔY75 as compared to control mice: 14.5±2.4 vs. 4.5±0.5 DCs/mm² respectively (n=4) (FIG. 4F). Similarly, the number of MHC class II⁺ cells with dendritic morphology was increased in KΔY75/KΔY75 skin, as compared to control skin: 32.0±7.0 vs. 10.8±2.5 DCs/mm² respectively (n=3). Staining of serial sections of the buccal mucosa with anti-MHC class II and anti-CD11b mAb revealed that, in contrast to control mice in which CD11b⁺ cells were barely detectable in the dermis, the majority of MHC class II⁺ DCs infiltrating the dermis of KΔY75/KΔY75 mice were CD11b⁺ cells. To further determine whether KARAP/DAP12 loss-of-function mutation affected the number of epidermal DCs in the skin, epidermal sheets from the ears were stained with anti-MHC class II, anti-DEC205 and anti-CD11c mAbs. All mAbs decorated a network of epidermal Langerhans cells of similar density in both control and mutant mice (FIGS. 4I and J). This result indicates that in the absence of a functional KARAP/DAP12 molecule, the abnormal accumulation of DCs in pluristratified epithelial tissues, such as the buccal mucosa and the skin, affected primarily the dermis. No significant changes in the distribution and density of DCs in spleen and in peripheral lymph nodes could be detected in KΔY75/KΔY75 mice. Taken together, these data indicate that the KARAP/DAP12 loss-of-function mutation results in the accumulation of DCs (and possibly pre-DCs) of myeloid origin in mucosal tissues, without detectable changes in distribution or phenotype of DCs in secondary lymphoid organs.

Phenotype and Function of Bone Marrow-Derived DCs in KARAP/DAP12 Knock-In Mice

The selective accumulation of DCs observed in KΔY75/KΔY75 mice might be the consequence of abnormal DC differentiation. To directly test this hypothesis, DCs were differentiated in vitro from bone marrow progenitors in the presence of GM-CSF. Flow cytometric analysis of day 7 bone marrow-derived DCs (BM-DCs) from control and KΔY75/KΔY75 mice, revealed a similar phenotype consisting of 50 to 60% CD11c⁺CD11b⁺ cells, characteristic of myeloid DCs, expressing low levels of DEC205, CD80 and CD86 (FIG. 5A, upper panels). Double staining of BM-DCs confirmed that both CD11c⁺ DCs and CD11b⁺ DCs co-expressed MHC class II molecules. Moreover, 24 hr LPS-stimulation of BM-DCs induced similar maturation of DCs from both mutant and control mice as shown by up-regulation of cell surface expression of MHC class II, DEC205, CD80 and CD86 molecules (FIG. 5A, lower panels). To determine whether DCs differentiated from KΔY75/KΔY75 mice were functional, we next investigated the ability of BM-DCs prepared from either control and KΔY75/KΔY75 mice to stimulate proliferation of allogeneic CD4⁺ T cells. As shown in FIG. 5B (left panel), as few as 4×10³ BM-DCs from either control or KΔY75/KΔY75 mice stimulated a strong proliferative response of allogeneic CD4⁺ T cells. Moreover, LPS-induced maturation of BM-DCs of either control or KΔY75/KΔY75 mice, resulted in similar enhancement of their allostimulatory capacity (FIG. 5B, right panel). Thus, BM-DCs derived from KΔY75/KΔY75 mice exhibited a normal maturation pathway associated with potent allostimulatory property for naive CD4⁺ T cells, characteristic of DCs.

Function of Myeloid Epithelial DCs in KARAP/DAP12 Knock-In Mice

Epithelial myeloid DCs have the capacity to migrate to draining lymph node upon antigen capture in the epithelium (reviewed in Banchereau et al., 2000 Ann. Rev. Immunol. 18: 767-811 of which content is herewith incorporated by reference). To directly test the migratory capacity of skin DCs from KΔY75/KΔY75 mice to draining lymph nodes, KΔY75/KΔY75 mice and +/+ littermates were skin-painted with the fluorescent hapten FITC. In control mice, 7-12% of FITC⁺ cells were found in the draining lymph nodes 24 hr after skin painting with FITC. The draining lymph node of KΔY75/KΔY75 mice, contained 8-25% of FITC⁺ cells. Double staining of lymph node cells for MHC class II or CD11b showed that in both control and KΔY75/KΔY75 mice, all FITC⁺ cells present in the draining lymph node were MHC class-II^{high} and CD11b⁺. Thus, the migratory capacity of skin DCs appeared normal in KΔY75/KΔY75 mice.

Contact sensitivity (CS) is a T-cell mediated inflammatory skin reaction in response to topical application of haptens, initiated by hapten capture by skin DCs, which migrate to the paracortical area of draining lymph node and prime hapten-specific T cells. Challenge of hapten-sensitized mice with the same hapten at a remote skin site (ear), induces recruitment of hapten-specific CD8⁺ effector T cells, which initiate the inflammatory reaction and edema of the skin. Thus, CS to 2,4-dinitrofluoro-benzene (DNFB) was used to investigate the in vivo function of skin DCs from KΔY75/KΔY75 mice. CS to DNFB was severely impaired in KΔY75/KΔY75 mice, as compared to control mice (FIG. 6). These results thus suggest that skin DCs from KΔY75/KΔY75 mice are unable to prime hapten-specific CD8⁺ T cells responsible for the CS response. Moreover, CS to DNFB develops similarly in C57B1/6, 129 and Balb/c excluding a mixed genetic background effect in the CS impairment observed in KΔY75/KΔY75 mice.

Discussion

ITAM-Dependent NK Cell Natural Cytotoxicity

In mouse NK cells KARAP/DAP12 dimers associate with activating Ly49 molecules (e.g. Ly49D, Ly49P). NK cells express CD3ζ and FcRγ polypeptides, and it has been described that Ly49D may associate with CD3ζ in transfected cell lines. Our results show that no activation signal through Ly49D could be detected in KΔY75/KΔY75 mice, indicating that Ly49D only functionally associates with KARAP/DAP12 in vivo. Together with the drastic impairment of NK natural cytotoxicity in CD3ζ-FcRγ^{-/-} mice, our results on KΔY75/KΔY75 mice reveal the importance of ITAM-bearing molecules in natural cytotoxicity. In NK cells, antibody-dependent cell cytotoxicity (ADCC) is mediated via CD16 associated with CD3ζ and/or FcRγ. Both ADCC and natural cytotoxicity are thus dependent upon ITAM-bearing polypeptides on NK cells, providing the basis for the pharmacological inhibition of ADCC and natural cytotoxicity by PTK inhibitors.

ITAM-bearing polypeptides couple the engagement of associated cell surface receptors to PTK-dependent pathways. When phosphorylated on ITAM tyrosine residues, ITAM-bearing polypeptides recruit the SH2-tandem PTKs, Syk and/or ZAP-70 (reviewed in Chu et al., 1998 Immunol. Rev. 165: 167-180 of which content is herewith incorporated by reference). In vitro studies have shown that phosphorylation of both tyrosine residues present in ITAM are mandatory to the recruitment and activation of Syk and ZAP-70. These results are supported by the low affinity of each SH2 domains for single tyrosine phosphorylated hemi-ITAM, as well as by the spatial arrangement of the N- and C-terminus SH2 domain of ZAP-70 which precisely fits the length of the spacer between each tyrosine residues in ITAMs (Chu et al., 1998 cf. supra). Along this line, we previously reported that mutation of a single tyrosine in KARAP/DAP12 ITAM prevents KARAP/DAP12-dependent activation of transfected cell lines (WO 98/49292). Our present data provide formal evidence that both tyrosine residues in KARAP/DAP12 ITAM are required for KARAP/DAP12 transduction function. These results thus support the critical role of SH2-tandem PTKs Syk and/or ZAP-70 as single effector molecules downstream of KARAP/DAP12. The role of Syk as a preferential downstream effector molecule for KARAP/DAP12 has been reported (McVicar et al., 1998J. Biol. Biochem. 273: 32934-32942 of which content is herewith incorporated by reference). In addition, Syk has been shown to be mandatory for NK cell natural cytotoxicity in cell lines (Brumbaugh et al., 1997J. Exp. Med. 186: 1965-1974 of which content is herewith incorporated by reference). However, NK cells derived from Syk^{-/-} or ZAP^{-/-} mice as well as from ZAP-deficient patients have been reported to be fully competent for natural cytotoxicity (Colucci et al., 1999 J. Immunol. 163: 1769-1774; Elder et al., 1994 Science 264: 1596-1599; Negishi et al., 1995 Nature 376: 435-438). Taken together, these results strongly suggest that in vivo Syk and ZAP-70 sub-serve redundant functions in NK cells. Formal demonstration of this compensatory signaling pathways will require the generation of Syk-ZAP^{-/-} mouse NK cells.

Another direct consequence of our findings is that NK cell surface molecules involved in NK cell cytotoxic function (e.g. CD2, DNAM-1) which are not physically associated with ITAM-bearing polypeptides might rather serve as co-receptors for NK cell cytotoxicity than as initiating NCRs, as it has been recently reported for 2B4 (Sivori et al., 2000 Eur. J. Immunol. 30: 787-793). The functional link between CD2 engagement and CD3ζ tyrosine phosphorylation supports this hierarchical organization of NK cell surface receptors. Similar conclusions may be drawn for mouse LAG-3. Although not detected on human NK cells, LAG-3 is expressed on mouse NK cells, and NK cells from LAG-3^{-/-} mice present deficient natural cytotoxicity towards selected tumor cell lines including the macrophagic J774 and IC-21 cell lines (Miyazaki et al., 1996 Science 272: 405408 of which content is herewith incorporated by reference). NK cells from KΔY75/KΔY75 mice are also deficient in their natural cytotoxicity towards J774 and IC-21 cell lines. A direct physical interaction between LAG-3 and KARAP/DAP12 in mouse NK cells cannot be ruled out, but would be unexpected due to the absence of transmembrane charged amino-acid residues in LAG-3. Therefore, our results suggest that LAG-3 might serve as a KARAP-dependent co-receptor for natural cytotoxicity in mouse NK cells.

Shaping of the NK Cell Repertoire of Ly49 Molecules

During NK cell development, a MHC-dependent education operates for the formation of the Ly49 repertoire. It has been thought that if MHC-specificities are similar for activating and inhibitory Ly49 isoforms, then activating Ly49 molecules may serve in a MHC-dependent selection. All known activating isoforms of classical and non-classical MHC class I receptors (Ly49 and CD94/NKG2 molecules) associate with KARAP/DAP12. Therefore, KΔY75/KΔY75 mice represent loss-of-function mutants for all activating MHC class I receptors. The shaping of the NK cell repertoire of MHC class I receptors appears unaltered in KΔY75/KΔY75 mice, as judged by mAb staining, as well as by the capacity of KΔY75/KΔY75 NK cells to distinguish RMA cell lines from their MHC class I⁻ variants (RMA/S). Our present results thus strongly suggest that the shaping of the NK cell repertoire of inhibitory MHC class I receptors does not depend upon the function of their activating isoforms. Consistent with these data, it has been recently shown that Ly49D activating molecules are expressed later in NK cell development than the inhibitory receptors (Smith et al., 2000 J. Exp. Med. 191: 1341-1354).

Myeloid Abnormalities in Absence of Functional KARAP/DAP-12

DCs represent an unique APC type by their ability to prime naive T cells after antigenic capture from peripheral tissues and migration to draining lymph node (Banchereau et al. 2000 cf. supra). In the small intestine of wild type mice, CD11c⁺ DCs reside in three distinct anatomical sites. Few DCs are present in the lamina propria of the villi. DCs also form a dense layer beneath the SED of Peyer's patches. Finally DCs can be found as mature interdigitating cells in the IFR of Peyer's patches (Kelsall and Strober 1996 cf. supra). Recent studies showed that the CD11c⁺DEC205⁻ DCs which are located in the SED region of Peyer's patches and in the lamina propria are CD11b⁺ myeloid DCs, while CD11c⁺ DEC205⁺ DCs within the IFR are CD8αα⁺ lymphoid DCs (Iwasaki and Kelsall 2000 cf. supra). SED DCs as well as lamina propria DCs may capture oral antigens translocating through the M-cell rich epithelium of Peyer's patches or through absorptive villus epithelial cells respectively, and activate T cells either locally in the Peyer's patches or after migrating through mesenteric lymph in the draining mesenteric lymph nodes.

KARAP/DAP12 is present in DCs, including BM-DCs and lack of signaling through KARAP/DAP12 ITAM causes a dramatic accumulation of DCs which reside in lympho-epithelial tissues. In the intestine, KΔY75/KΔY75 mice exhibited a major increase in the number of CD11c⁺DEC205⁻ DCs in the lamina propria of the mucosal villi, as well as in the SED, but not in the IFR of Peyer's patches. The recent finding that CCR6-deficient mice exhibit a selective defect in the CD11c⁺CD11b⁺ subset of DCs in the SED of Peyer's patches (Cook et al. 2000 Immunity 12: 495-503), responsive to MIP-3α produced by the dome epithelium (Iwasaki and Kelsall 2000 cf. supra), suggests that KARAP/DAP12 may be involved in DC responsiveness to chemokines produced by mucocutaneous epithelia. Lack of functional KARAP/DAP12, also results in abnormally high numbers of MHC class II⁺ and of CD11b⁺ cells with dendritic morphology in skin and buccal mucosa dermis. These cells could be either myeloid DCs, or activated monocytes/macrophages entering the dermis from the blood. Indeed, activated monocytes extravasating through endothelial cells may either become resident macrophages or differentiate into DCs upon emigration from the tissue through afferent lymph (Randolph et al., 1998 Science 282: 480-483; Randolph et al. 1999 Immunity 11: 753-761 of which contents are herewith incorporated by reference).

Concluding Remarks

Taken together, our data strongly support the hypothesis as which signaling through KARAP/DAP12 plays a critical role in the differentiation and/or activation programs of myeloid DCs/pre-DCs within epithelia. The cell surface receptors associated with KARAP/DAP12 which are responsible for these pathways remain to be identified and may include SIRPβ1, MDL-1, TREM-1 or TREM-2. It will also be important to investigate whether KARAP/DAP12-deficient patients, which develop the Polycystic Lipomembranous Osteodysplasia with Sclerosing Leukoencephalopathy (PLOSL) (Paloneva et al., 2000 Natl; Genet. 25: 357-361 of which content is herewith incorporated by reference) present abnormalities within the myeloid compartment and whether such alterations are related to the PLOSL pathogenesis. Finally, the impaired CS to DNFB in KDY75/KDY75 mice suggests an inadequate priming of hapten-specific CD8⁺ T cells (Tc1 effector) mediating tissue inflammation (Kehren et al. 1999 L. Exp. Med. 189: 779-786). Indeed, IL-12 secretion by myeloid DCs is mandatory for priming IFN-γ secreting CS effector T cells and for the development of the skin inflammatory response (Müller et al. 1995 J. Immunol. 155: 4661-4668). Along this line, KARAP/DAP-12 deficient mice also fail to develop the Th1-mediated experimental auto-immune encephalitis induced by myelin oligodendrocyte glycoprotein peptide immunization (Bakker et al., 2000 cf. supra). It thus remains to determine whether and how the accumulation of myeloid DCs/pre-DCs in epithelia is linked to the deficient Tc1 and Th1 priming observed in the absence of a functional KARAP/DAP12 signaling pathway.

### EXAMPLE 2

The procedure is a modification of Mendel, I. et al. 1995 (A myelin oligodendrocyte glycoprotein peptide induces typical chronic experimental autoimmune encephalomy-elitis in H-2 b mice: fine specificity and T cell receptor V g expression of encephalitogenic T cells. Eur. J Immunol. 25: 1951-1959), of which content is herewith incorporated by reference.

Induction of EAE (Experimental Auto-Immune Encephalomyelitis)

EAE were obtained by a single injection, s.c. at one site on the flank, of 0.2 ml of emulsion composed of 200 μg pMOG 35-55 (Syntem, Nimes, France) in complete Freund's adjuvant (CFA) supplemented with 500 μg of Mycobacterium tuberculosis (Mt) H37Ra (Difco Lab., Detroit, Mich.). Mice as described in the above example 1 received, the same day, 500 ng of Pertussis toxin (PT) (List biological Laboratories, Inc Cambell, Calif.) in 0.2 ml PBS in the tail vein. The mice were observed daily and clinical manifestations of EAE recorded on a scale of 0-6: (1: tail paralysis; 2: slight weakness of the hind limb; 3 partial hind limb paralysis; 4: complete hind limb paralysis; 5 total paralysis of hind and forelimbs; 6: moribund or death). The results are given in Table 1:

TABLE 1

| | | | Maximum | Clinical score |
|---|---|---|---|---|
| Mice (sex) | EAE | Onset | clinical score | J40 |
| (fem) −/− | − | — | 0 | 0 |
| (fem) −/− | − | | 0 | 0 |
| (fem) −/− | + | J14 | 1.5 | 0 |
| (fem) −/+ | − | — | 0 | 0 |
| (fem) −/+ | + | J16 | 4 | 3 |
| (fem) −/+ | + | J17 | 4 | 4 |

These results clearly show that mice that bears a functional copy of KARAP (∓) are far more susceptible to EAE induction as compared to mice that have no functional copies of KARAP (−/−).

### EXAMPLE 3

Animals Over-Expressing KARAP

Construction of Human KARAP Transgenic Mice.

The complete cDNA of human KARAP (WO 98/49292) has been cloned (Sall/BamHI) downstream of the mouse class I promoter H-2Kb, and upstream of a polyA signal and Ig enhancer (FIG. 7), as described previously (Pircher, II., et al. 1989 EMBO J. 8: 719-727). This construction has been injected into C57BL/6 (B6) (H-2^{b/b})×CBA/J (H-2^{kk}) F₂ fertilized eggs. Transgenic lines were established and maintained by crossing of founders with B6 mice. Transgenic founder mice and their progeny were identified by PCR on RNA isolated either from spleen and thymus using human KARAP specific oligonucleotides (FIG. 8b).

Number of copies were estimated by southern blot analysis using a radiolabeled human KARAP specific probe (complete cDNA of human KARAP). Mice potentially containing 5, 11, and 30 copies of human KARAP were selected for further studies (FIG. 8a). Expression of human KARAP protein was confirmed by Western Blot analysis with antiserum against human KARAP (FIG. 8C see below).

Generation of Anti Human KARAP Antiserum.

Antiserum against human KARAP was obtained by immunization of rabbit with a synthetic peptide reproducing the intracytoplasmic tail of human KARAP (sequence : ITETESPYQELQGQRSDVYSDLNTQR). The anti serum has been purified on beads coupled with the above peptide.

Antibodies and Flow Cytometry.

Flow cytometry was performed as described for knock-in mice in the above example 1. Antibody GR-1 can be obtained from Pharmingen.

Phenotype of KARAP Transgenic Mice.

Lethality of the Transgene

FIG. 9 shows the rate of death of heterozygote animal that bear 30 copies of the transgene where more than 50% of animal are dead after 28 weeks, versus 0 for wild type animals. Animals having integrated lees copies of the transgene are far less susceptible to increase death rate.

This lethality is associated with massive accumulation of cells in the lungs of the animal. Immunohistochemical experiments show that the infiltrated cells are of myeloid origin (macrophages and small number of granulocytes) and stain positive with the KARAP antiserum.

Analysis of the Lymphoid Compartment.

The lymphoid compartment has been studied both in the thymus and the spleen.

Analysis by flow cytometry using anti antibodies against CD3, CD4, CD8, B220, and DX5 show that there are strong defect in heterozygote mice with 30 copies of transgene: Lymphoid development (FIGS. 10A and 10B) as the total number of cells is decreased in the thymus of transgenic animals as compared to wild type. This phenomena correlates with increase of CD4-, CD8-cells and decrease in single positives (CD4+, CD8+) and double positive (CD4+,CD8+).

Total number of cells in the spleen is greatly reduced in transgenic animal (FIG. 11A), and farther analysis of T cells (CD3+), NK (DX5+, CD3−) cells and B cells (B220+) show that cell numbers of these cell types are dramatically reduced in transgenic animals.

The overall intensity of the defect in the spleen and thymus is directly related to the number of copies of KARAP integrated in the genome of the mice (FIGS. 10A, 10B, 11A, 11B).

Analysis of the myeloid cells in the peripheral blood of transgenic animals shows that cells of this compartment are greatly enriched in GR1 positive and Mac-1 positive cells in transgenic animals as compared to wild type animals (FIG. 12). This shows that the above lymphopenia is accompanied by an increase in myeloid cells.

### EXAMPLE 4

KARAP Interaction Motif

Several biochemical observations revealed that KARAP shares striking similarities with members of the ITAM bearing polypeptide family like CD3ζ, CD3ε, FcεRI-γ KARAP associate with KAR which contains a charged aminoacid residue in their transmembrane portion, similarly to ITAM bearing polypeptides and their associated receptors (e.g. TCR, BCR, FcγRI, Fcγ Receptors). It may be difficult to disrupt selectively KARAP function because of these structural similarities.

A close look at the charged amino acid residues in the transmembrane regions of ITAM bearing molecules provides a structural basis for disrupting selectively KARAP association with its KAR associated molecules.

Transmembrane regions, constituted by hydrophobic stretch of aminoacids, are thought to be alpha helical. The charged amino acid (D residue) of the KARAP molecule is in the middle of the transmembrane region, whereas ζ and FcγRI γ are in a region thought to be closer to the surface of the cell, as shown in the following alignment of transmembrane region of the three molecules.

Extracellular region Transmembrane region Intra cellular region FcERIγ LCYILDAILFLYGIVLTLLYC CD3ζ LCYLLDGILFIYGVILTALFL human KARAP GVLAGIVMGDLVLTVLIALAV (from position 41 to position 61 on mature human KARAP, i.e. corresponding to positions 173-235 of SEQ ID No. 31 in WO98/49292) mouse KARAP GVLAGIVLGDLVLTLLIALAV (from position 43 to position 63 on mature mouse KARAP, i.e. from position 36 to position 57 of SEQ ID No. 17 in WO 98/49292—on FIG. 16 of WO 98/49292: from position 61 to position 81 of SEQ ID No. 17 alignment-)

Molecules that could interact with the charged residue of KARAP can thus easily designed to avoid the interaction with the charged residue of ζ and γ on this basis.

Preferred inhibitory compounds target aminoacid D in position 50 of human mature KARAP (position 52 on mouse mature KARAP, i.e. position 45 on SEQ ID No. 17 of WO 98/49292).

Receptors that associate with KARAP or CD3 ζ or FcRγ display also a charged amino acid in the transmembrane region. Again, receptors that associate selectively with ζ and γ present a charged amino acid (K or R residue) within the transmembrane domain which is close to the junction with extracellular domain, whereas receptors selectively associated with KARAP display their charged amino acid which are central within the transmembrane domain, as show by the following alignment:

Transmembrane regions or receptors associated with KARAP Extracellular region Transmembrane Intra cellular region

| | |
|---|---|
| SIRPβ1 | LLVALLGPKLLLVVGVSAIYIC |
| KIR2DS1V | LIGTSVVKIPFTILLFFLL |
| NkP44 | IALVPVFCGLLVAKSLVLSALLV |
| TREM1 | FNIVILLAGGFLSKSLVFSVLFA |
| TREM2 | TSILLLLACIFLIKILAASALWA |
| HNKG2C | FPILVITKLVTAMLVICIIGLV |
| LY49D | TMLVAVTVLRLSILIGLAIVIL |
| MDL1 | LLFFTMGVVKIVVVILGSIIMHW |

Transmembrane regions of receptors associated with ζ or γ Extracellular Transmembrane Intra cellular region

| | |
|---|---|
| PIR-A1 | LIRMGMAVLVLIVLSILAT |
| ILT1 | LIRMGVAGLVLVVLGILLF |
| hNKp46 | LLRMGLAFLVLVALVWFLV |
| hNKp30 | LLRAGFYAVSFLSVAVGST |
| mNKRP1A | LVRVLVSMGILTVVLLILGACSL |

Molecules that could interact with the charged residue of KARAP could be designed to avoid the interaction with the charged residue of ζ and γ on this basis.

### EXAMPLE 5

Method for the Identification of Compounds Capable of Inhibiting a KARAP-Mediated Immune Response

Selection of Starting Compounds to Test:

Several combinatorial chemistry libraries or natural compound libraries are commercially available. These libraries consist of thousand of purified compounds that are structurally diverse and whose molecular structure is known or can be obtained by molecular modeling. Such chemical libraries can be obtained from companies such as CEREP or BioFocus. These companies are cited as possible sources and should be in no event considered as limitative.

Molecular modeling can be performed on the compounds of such libraries to search for compounds with hydrophobic stretch of different length that could insert in the plasma membrane of a cell and having polar portion susceptible to interact with a charged membrane residue of a membrane protein.

Either complete combinatorial library or compounds selected with the above criteria are tested in in vitro assays for screening of compounds selectively inhibiting KARAP transduction.

Test for Inhibition of KARAP Transducing Signal:

As described above, the screening method should allow to discriminate compounds that selectively inhibit KARAP transduction without inhibiting other ITAM bearing transducing compound such as ζ or ε.

A preferred assay consists in a cell based assay based on Rat basophilic Leukemia cells (RBL-2H3, ATCC n^{o} CRL2256), transfected with KARAP and a KAR that transduces via KARAP, for example P50.3, where an antibody is available commercially (GL183, Beckman Coulter). RBL-2113 constitutively express the high affinity IgE receptor that transduces signal through constitutively expressed FceRI γ molecule. Tranduction through either KARAP or γ results in the degranulation of the cells that can be measured by histamin and serotonin release.

Crosslinking of IgE receptor can be done by cultivating the cells on 96 well plates coated with IgE monoclonal antibody such as LO-DNP-30 (commercially available from Harlan bioproducts for science, cited for example).

Cross linking of KAR can be done by cultivating the cells on 96 well plates coated with Fab′2 fragment of GL183 antibody.

The compounds to test will be put in the culture plates and serotonin or histamin will be measured by commercial kits (Beekmancoulter for example).

Compounds will be selected on their ability to inhibit serotonin release when KAR is crosslinked and not to inhibit serotonin release when IgE receptor is crosslinked.

Another method comprises the preparation cell membrane by lysis of the cells with known mild detergent such as digitonin and the testing in a sandwich assay the physical association of the KAR with KARAP, or on the other hand of FcεRI with gamma. GL183 and IgE monoclonal coated beads (such as dynal) beadscan be used on one side. Bound KARAP or γ can be revealed by phosphorylation with incorporation of radiolabeled 32P (Amersham) by commercial kinase such as recombinant lck. Test compound are selected through their capacity of inhibiting P32 incorporation on GL183 coated beads and not on IgE coated beads, therefore showing selective inhibition of association of KARAP with its KAR.

Test of Compounds in vivo:

Previously selected compounds are tested in vivo either by injection or orally for their capacity:

to inhibit contact sensitivity in normal mice to levels obtained with KARAP deficient mice, and/or

to inhibit auto-immune disease models such as EAE in normal mice to levels obtained with KARAP deficient mice, and/or

to have no side effect either immunological or non immunological on KARAP deficient mice, and/or

to increase life of KARAP over-expressing mice to levels comparable to normal mice, and/or

to increase lymphocyte populations of KARAP over-expressing mice to level comparable to normal mice, and/or

to decrease myeloid population of KARAP over-expressing mice to level comparable to normal mice.

The results showing that KARAP/DAP12-deficient mice exhibit an accumulation of DC in muco-cutaneous epithelia, associated with an impairment of hapten-specific contact sensitivity as well as a resistance to develop experimental autoimmune encephalomyelitis evoke inadequate in vivo T cell priming in KARAP/DAP12 loss-of-function mutant mice and suggest that KARAP/DAP12-driven signals might be required for optimal antigen-presenting cell function and/or inflammation, two major functions ensured by cells of the innate immune system. This hypothesis is consistent with the cellular distribution of KARAP/DAP12 polypeptides. Indeed, KARAP/DAP12 is expressed on all detectable neutrophils as well as on the vast majority of monocytes/macrophages and NK cells in spleen (FIG. 13A). To perform intracellular flow cytometric assays, cells from spleen were isolated as previously described. Before staining by flow cytometry, Fc receptors (FcγRII/III) were blocked using either 5% normal mouse serum or rat anti-mouse CD16/CD32 (2.4.G2) for 30 min on ice. Cells were first extracellulary stained using the following mAbs: fluorescein isothiocyanate (FITC)-conjugated anti-CD3ε, phycoerytbrin (PE)-conjugated NK1.1, PE-conjugated anti-TCRβ chain, biotin-conjugated anti-TCRγζ, PE-conjugated anti-CD19, PE-conjugated Ly-6G (Gr-1) obtained from Becton Dickinson-PharMingen, FITC-conjugated anti-CD11b obtained from Beckman Coulter and allophycocyanin (APC)-conjugated streptavidin obtained from Caltag. Intracellular KARAP/DAP12 staining was then performed on cells fixed using PBS containing 4% paraformaldehyde for 10 min at room temperature and permeabilized/stained using PBS containing 1% BSA, 1% saponin as well as rabbit anti-DAP12 polyclonal antibody. Antibody staining was revealed by a FITC-conjugated anti-rabbit polyclonal antibody (Beckman Coulter). Cells were analyzed on a FACScalibur apparatus using CellQuest software (Becton Dickinson).

KARAP/DAP12 is also expressed on a fraction of γζ T cells (16.1±7%). In contrast, KARAP/DAP12 is barely detectable in splenic CD4⁺ αβ T cells, CD8⁺ αβ T cells, follicular B220⁺CD23⁺ B cells and marginal zone B220⁺CR1⁺CD23⁻ B cells (FIG. 13A). Thus, within the hematopoietic compartment, KARAP/DAP12 polypeptides are preferentially expressed in cells belonging to the innate immune system including both myeloid cells and non-conventional lymphocytes.

In order to pursue the analysis of KARAP/DAP12 function in vivo, experiments were carried out generate KARAP/DAP12 gain-of-function mutant mice. Aggregation of ITAM-bearing polypeptides is critical for the initiation of downstream signaling event.

It was then assumed that over-expression of wild type KARAP/DAP12 polypeptides in a transgenic mouse model would increase the likelihood of KARAP/DAP12 homophilic interaction and could therefore represent a transgene dose-dependent model of gain-of-function mutants. Consistent with the high degree of homology with the mouse orthologs, human KARAP/DAP12 polypeptides are functional in mouse cells, but can be discriminated from endogenous mouse KARAP/DAP12 polypeptides by the use of anti-human KARAP/DAP12-specific antibodies. The inventors therefore generated KARAP/DAP12 transgenic mice (Tg-hKARAP mice) using the full length human KARAP/DAP12 cDNA under the control of a transgenic cassette that drives a broad hematopoietic expression.

For generation of Tg-hKARAP, human KARAP/DAP12 full length cDNA was prepared by RT-PCR from human RNA using the following primers: KARAP/DAP12.1 forward (5′-CCGCTCGAGCGGCTTCATGGGGGGACTTG-3′) containing a XhoI restriction site and KARAP/DAP12.1 reverse (5′-CGCGGATCCGCGGCTGACTGTCATGATTCG-3′) containing a BamHI restriction site. The PCR products were subcloned in the MHC class I promoter/immunoglobulin enhancer expression cassette pHSE3-XhoI (37) using 5′ SalI and 3′BamHI restriction sites. The construction was injected into fertilized C57BL/6 (H-2^{b}) X CBA/J(H-2^{k}) F₂ eggs. Transgenic founder mice and their transgenic progenies were identified by PCR with primers specific for human KARAP/DAP12 cDNA using the following primers:

KARAP/DAP12.2 forward
(5′-ATGGGGGGACTTGAACCCTGC-3′) and
KARAP/DAP12.2 reverse
(5′-GTATCATGTTGCTGACTGTCA-3′).

Transgenic lines were established and maintained by crossing of founders with C57BL/6 mice. Unless indicated, all the mice used in this study were between 6 and 10 weeks old and were maintained at the Animal Facility of the Centre d'Immunologie de Marseille-Luminy. The H-2K^{b} promoter/Igμ enhancer cassette drives an early expression of the transgene in bone marrow cells and therefore leads to transgene expression in both lymphoid cells and myeloid cells.

Ten transgenic founder mice were obtained and Southern blot analysis revealed distinct integration of 1 to 30 human KARAP/DAP12 cDNA copies in these animals.

Extensive analyses were performed on 3 independent transgenic lines Tg-hKARAP11, Tg-hKARAP17 and Tg-hKARAP30 (with 11, 17 and 30 integrated copies respectively), established following stable transmission of the human KARAP/DAP12 transgene.

The expression of human KARAP/DAP12 polypeptides was detected in splenocytes prepared from Tg-hKARAP mice by immunoblotting using an anti-human KARAP/DAP12-specific goat antiserum.

Immunoblot analysis was performed onto 12% polyacrylamide gel. Proteins were electrophoresed under denaturing conditions and electroblotted to nitrocellulose membranes using a Trans-Blot Semi-Dry (Bio-Rad Laboratories). Membranes were blocked overnight with 5% non-fat dry milk in PBS and then incubated for 2 h with rabbit anti-human KARAP antiserum diluted 1:400 in PBS plus 5% nonfat dry milk. Membranes were washed with PBS Tween 0,05% and incubated for 1 h with horseradish peroxidase-conjugated goat anti-rabbit polyclonal (Sigma) secondary Ab diluted 1:16000 in PBS Tween 0,05%. Proteins were detected using enhanced chemiluminescence reagents (ECL Plus; Amersham Pharmacia Biotech).

The expression was transgene dose-dependent as assessed by the progressive increase in the amount of hKARAP/DAP12 polypeptide with increasing transgene copy number (FIG. 13B).

The hematopoietic compartment in Tg-hKARAP mice was analyzed and bone marrow, thymus, peripheral blood and spleen isolated from Tg-hKARAP and non-transgenic control littermates for expression of cell lineage markers by flow cytometry were compared.

The results are given in Table 2:

TABLE 2
Hematological parameters in KARAP/DAP12 transgenic mice. Cells were
prepared from indicated origins (Tg-hKARAP 30 and control non transgenic
littermates, n = 3 to 10), and enumerated upon total cell counting and flow cytometric
analysis of indicated cell compartments. Results are expressed as mean values ± SEM.
(nd) indicates not done. Asterisk indicates statistically significant difference between
Tg-hKARAP30 and control mice (*P < .05; **P < .005; ***P < .0005).

| | | | Bone |
|---|---|---|---|
| | Peripheral blood cells | Spleen cells | marrow cells |
| | (cells/μl) | (×10⁻⁶/spleen) | (×10⁻⁶/femur) |
| T Lymphocytes | | | |
| (CD3⁺ B220⁻) | | | |
| Control mice | 1930.0 ± 220.6 | 38.4 ± 4.9 | nd |
| Tg-hKarap30 mice | 64.5 ± 21.9*** | 2.9 ± 1.5* | nd |
| B Lymphocytes | | | |
| (CD3⁺ B220⁺) | | | |
| Control mice | 1821.0 ± 402.2 | 65.5 ± 8.2 | 3.2 ± 1.0 |
| Tg-hKarap30 mice | 61.9 ± 15.5* | 11.1 ± 4.4** | 0.7 ± 0.3* |
| NK Cells | | | |
| (CD3⁻ DX5⁺) | | | |
| Control mice | 468.3 ± 73.1 | 4.9 ± 0.7 | nd |
| Tg-bKarap30 mice | 77.5 ± 19.8** | 2.0 ± 0.6* | nd |
| Monocytes/ | | | |
| Macrophages | | | |
| (CD11b⁺ Ly-6G⁻) | | | |
| Control mice | 232.7 ± 60.5 | 7.7 ± 2.3 | 0.5 ± 0.1 |
| Tg-hKarap30 mice | 61.2 ± 11.7* | 6.8 ± 3.9 | 1.0 ± 0.4 |
| Neutrophils | | | |
| (CD11b⁺ Ly-6G⁺) | | | |
| Control mice | 516.5 ± 168.2 | 6.5 ± 1.7 | 6.5 ± 0.6 |
| Tg-hKarap30 mice | 9389.0 ± 1701.0** | 86.4 ± 57.0 | 15.4 ± 3.4 |
| Leukocytes | | | |
| Control mice | 4208.0 ± 425.3 | 140.4 ± 11.2 | 18.2 ± 2.5 |
| Tg-hKarap30 mice | 9733.0 ± 1656.0*** | 97.6 ± 60.3 | 23.0 ± 3.2 |

A first striking observation was the markedly decreased frequencies and absolute numbers of splenic and peripheral blood T, B and to lesser extent NK lymphocytes in Tg-hKARAP mice. Importantly, this lymphopenia was transgene dose-dependent as manifested by the progressive reduction in T, B and NK lymphocyte number with increasing transgene copy number (see supplemental material 1 herein below). A drastic reduction of B cells was also observed in bone marrow. A major impairment in B cell development was further confirmed by pre-B cell colony assays. After 7 days in culture with human recombinant IL-7, B220⁺ B cells were either barely or not detectable when bone marrow cells from Tg-hKARAP mice were used, in contrast to the vigorous expansion of B220⁺ generated from non-transgenic control littermates (see supplemental material 1 herein below). Similarly, the development of T cells was severely impaired in Tg-hKARAP mice as assessed by thymic cellularity: 250.0±30.2 vs. 48.4±14.7 vs. 19.3±4.2×10⁶ cells/thymus for control, Tg-hKARAP11 and Tg-hKARAP30 mice, respectively. This decrease in thymocyte numbers is accompanied with a drastic reduction of the size of the CD4⁺CD8⁺ double positive thymocyte subset (see supplemental material 1 herein below). The alteration in the development of conventional T and B lymphocytes in Tg-hKARAP mice was associated in spleen with a severe white pulp hypoplasia and with a transgene dose-dependent reduction of total immunoglobulin (Ig) G serum levels interesting IgM, IgG1 and IgG2a in the absence of controlled immunization. As expected, no OVA-specific Ig (IgM, IgG1, IgG2a) could be detected in Tg-hKARAP mice upon OVA immunization (see supplemental material 2 herein below).

Supplemented Material 1. T and B Cell Lymphopenia in Tg-hKARAP Mice (FIGS. 17 and 18)

A-B-D) Cells isolated from Tg-hKARAP11, Tg-hKARAP30 mice and non-transgenic littermates (control) were prepared from indicated tissues and analyzed by two-color flow cytometry for the cell surface expression of CD3ε and B220 (A, B) or CD4 and CD8α (D). The frequencies of each leukocyte sub-population are indicated in their respective quadrants. The study of the lymphoid organs was performed by FACS analysis using PE-conjugated anti-CD4, APC-conjugated anti-CD8α (Pharmingen) and antibodies previously described (14).

C) Bone marrow cells were cultured in the presence of rhIL-7 (10 ng/ml) in 1% methylcellulose containing medium (pre-B colony assay). After 7 days, colonies were mixed prior to cell counting and FACS analysis using anti-CD3ε and anti-B220, anti-CD11b and anti-Ly-6G mAbs. Results of one representative out of 3 independent experiments were expressed as the absolute number of pre-B cells (B220⁺CD3ε⁺) per bone marrow cultures.

Supplemented Material 2. Hypoglobulinemia in Tg-hKARAP Mice (FIG. 19)

A) Indicated Ig isotypes were measured by ELISA in sera from Tg-hKARAP11 mice (◯), Tg-hKARAP28 (Δ) and non-transgenic littermates (control, ●). Results of one representative out of a minimum of 4 independent experiments are expressed as O.D. as a function of serum dilutions.

Therefore, overexpression of KARAP/DAP12 polypeptides leads to a transgene dose-dependent impairment in the development of conventional αβ T and B lymphocytes, which is associated with a functional immunodeficiency. Alteration of lymphoid development has also been documented in transgenic mice overexpressing ITAM-bearing polypeptides related to KARAP/DAP12, such as CD3ε and FcRγ, suggesting that the transcription of genes encoding for these signaling molecules is tightly regulated during the course of lymphoid development.

A second feature of the hematological abnormalities observed in Tg-hKARAP mice concerns myeloid cells. A 3 to 5-fold reduction in the absolute number of peripheral blood monocytes was detected in Tg-hKARAP30 mice as compared to non-transgenic control littermates (Table 2). In contrast, a 13 to 18-fold increase in the absolute number of neutrophils was observed in peripheral blood from Tg-hKARAP30 mice (Table 2), as well as in spleen of most transgenic mice examined (FIG. 14A). Consistent with this neutrophilia, the size of the neutrophilic compartment was also increased in the bone marrow of Tg-hKARAP30 mice as compared to non-transgenic control littermates (FIG. 14B, Table 2). Neutrophilia was dependent upon the dose of human KARAP/DAP12 transgene (FIG. 14A) and upon the age of the mice (FIG. 14B). Neutrophilia has been described as the consequence of hematological neoplasia, such as chronic myeloid leukemia, or as the consequence of microbial infection. Colony-forming unit (CFU) assays for granulocytes were performed using bone marrow cells isolated from Tg-hKARAP and non-transgenic control littermates and showed no significant differences in the number of CFU-GM (CD11b⁺Ly-6G⁺) per bone marrow cultures (FIG. 14C).

To support the formation of myeloid colonies, bone marrow cells were cultured in Iscove's Modified Dulbecco's Medium (IMDM)-based methylcellulose media (Methocult 3534; Stem cell technologies) supplemented with 5 μg/ml of recombinant mouse Granulocyte Macrophage Colony Stimulating Factor (GM-CSF; R&D system, Inc). CFU-Granulocyte/Macrophage (CFU-GM), CFU-Granulocyte/Erytlrocyte/Macrophage (CFU-GEM) and CFU-Macrophage (CFU-M) were scored by counting individual colonies according to their morphology after 12 days of culture prior to flow cytometric analysis.

These data rule out the possibility that overexpression of KARAP/DAP12 polypeptide leads to an increased differentiation of progenitor cells into granulocytes. In the absence of detectable infection, these data suggest that the high neutrophil counts observed in Tg-hKARAP mice may be due to an enhanced secretion of growth factors and/or pro-inflammatory cytokines which have been shown to induce neutrophilia directly or indirectly. Consistent with this possibility, a large increase in the serum levels of G-CSF (FIG. 15A) and TNF-α (97±32.6-fold increase) was observed in Tg-hKARAP30 mice as compared to non-transgenic control littermates.

The serum levels of G-CSF were determined using a mouse specific enzyme-linked immunoabsorbent assay (ELISA) kit (R&D Systems, Inc), following the manufacturer's protocol. The serum levels of TNF-α were determined using a bioassay. In brief, TNF-α sensitive WEHI-3B clone 13 cells were cultured for 24 h in media in the presence or absence of transgenic or control mice sera and tested for their survival. Cell survival was assessed using MTT (Sigma).

Other signs of inflammation were observed in Tg-hKARAP30 mice. While young Tg-hKARAP30 mice showed no difference in size, and behavior compared with wild-type littermates, after 4-weeks of age, they progressively developed, with a nearly 100% penetrance, a wasting syndrome characterized by cachexia (total body weight: 11.5±0.4 g for Tg-hKARAP30 mice as compared to ˜20.5±1.1 g for non-transgenic control littermates) and premature mortality (FIG. 15B). This fatal outcome of KARAP/DAP12 overexpression was associated with a quasi-complete fat pad disappearance and a massive pulmonary inflammation which resembles desquamative interstitial pneumonia in humans. Indeed, histopathology analysis of lung sections revealed major alterations of the parenchyma with accumulation of large intra-alveolar cells of macrophagic type (FIG. 15C). Immunohistology of lung sections using anti-human KARAP/DAP12-specific antiserum revealed the positive KARAP/DAP12 staining of alveolar invading cells (FIG. 15C). This excessive inflammatory reaction evokes several features of post-infectious inflammation such as the appearance of multinucleated giant cells, often observed during the course of mycobacterial infection. However, Gram, Gomori-Grocott and Periodic Acid Schiff coloration failed to detect any pathogenic microbial infection in Tg-hKARAP30 tissues. Consistent with said in vivo data, signaling through KARAP/DAP12 in vitro induces marked morphological changes on mouse myeloblastic leukemic cell transfectants such as the apparition of multinucleated giant cells. Thus, the apparently spontaneous inflammatory syndrome that develops in Tg-hKARAP30 mice emphasizes the central role of KARAP/DAP12 polypeptides in the development of inflammatory reactions in the absence of detectable microbial infection.

The cross-talk between KARAP/DAP12-dependent pathways and infectious signals in vivo was also investigated. Tg-hKARAP11 mice, which do not develop any spontaneous wasting syndrome were used. Tg-hKARAP mice and non-transgenic control littermates were challenged using a model of experimental septic shock induced by intra-peritoneal injection of Escherichia coli lipopolysaccharide (LPS). As shown in FIG. 16, overexpression of KARAP/DAP12 polypeptides leads to LPS hyperresponsiveness and to premature death, as compared to the progressive death of non transgenic littermates.

The in vivo analysis of KARAP/DAP12 transgenic mice thus reveals that overexpression of KARAP/DAP12 polypeptides leads to T and B lymphopenia, to neutrophilia associated with a fatal inflammatory syndrome which apparently develop spontaneously, as well as to an increased sensitivity to LPS-induced mortality. KARAP/DAP12 polypeptides are not detected in most conventional lymphocytes. Therefore, the lymphoid abnormalities observed in Tg-hKARAP mice do not reflect any endogenous role of KARAP/DAP12 in lymphoid development. In addition, the lymphopenia and the development of the inflammatory syndrome appear independently in mice overexpressing KARAP/DAP12. Indeed, the inflammatory syndrome develops mostly in Tg-hKARAP30, whereas lymphopenia is readily observed in transgenic mice expressing 2 copies of the transgene. Moreover, other mutant mouse models of T- and/or B-immunodeficiency (such as CD3ζ^{-/-}, CD3ζε^{-/-} and RAG-1^{-/-} mice) do not develop a wasting inflammatory with lung infiltration, in the same housing environment. The main phenotype of Tg-hKARAP mice is thus restricted to their myeloid abnormalities, which bring novel insights into the role of KARAP/DAP12, as an ITAM-bearing transduction polypeptide, in pro-inflammatory innate immune responses.

Several receptors require association with KARAP/DAP12 for optimal cell surface expression. A possible explanation for the phenotypes observed in KARAP/DAP12 transgenic mice thus resides in the up-regulated surface expression of KARAP/DAP12-associated receptors, leading to their increased engagement. However, using mAbs directed against Ly49D, Ly49H, NKG2C and TREM-1 mouse receptors, no significant increase in the cell surface expression of such KARAP/DAP12-associated receptors was detected. These data support the hypothesis that KARAP/DAP12 transgenic mice represent a transgene dose-dependent model of gain-of-function mutants, and that the transgene dose-dependent phenotypes observed in KARAP\DAP12-transgenic mice are the results of a constitutive activation of KARAP/DAP12-dependent pathways which occurs independently of the engagement of associated receptors.

The hyperresponsiveness of KARAP/DAP12 transgenic mice to E. coli LPS injection suggest a model as which KARAP/DAP12-driven pathways act as co-stimulatory signals for pro-inflammatory innate immune responses in the presence of microbial challenge. This hypothesis is supported by the in vitro potentiation of LPS stimulation mediated via KARAP/DAP12 on mouse myeloblastic leukemic cell transfectants (up-regulation of CD11b, MHC class II, CD86 and CD11c), as well as on monocytes and neutrophils via TREM-1 engagement for the production of inflammation mediator (e.g. interleukin-8, monocyte chemoattractant protein-1, TNF-α). More importantly, in vivo blocking of TREM-1 by injection of TREM-1/IgG1 fusion proteins has been reported to protect mice from lethality induced by LPS, E. coli peritonitis as well as polymicrobial sepsis caused by caecal ligation and puncture. Yet, no protection of KARAP/DAP-12 loss-of-function mice against LPS-induced mortality could be detected. A possible interpretation of this result, is that multiple receptors are associated with KARAP/DAP12 on cells involved in the complex cascade of events which follow LPS injection in vivo. It is thus possible that the functional deficiency of receptors other than TREM-1 might be detrimental to the control of the inflammatory response to LPS. Nevertheless, KARAP/DAP-12 loss-of-function mice are hyporesponsive to antigen-specific immunization in the presence of microbial adjuvant. Taken together these in vitro and in vivo results indicate that KARAP/DAP12 amplifies the activation signals generated by the recognition of microbes or microbial products. The pattern recognition receptors involved in the recognition of infectious signals, such as the Toll-like Receptors (TLR), are primarily described as inducers of NF-κB as well as of activators of c-Jun NH2-terminal kinase (Jnk) and p38 mitogen-activated protein kinase (MAPK). KARAP/DAP12-driven pathways initiate a protein tyrosine kinase-dependent signaling pathway which activates the ERK1 and ERK2 MAPK, leading in part to the activation of fos/jun transcription factors. Therefore, KARAP/DAP12-dependent pathways may synergize with TLR-dependent triggering by providing the activation of a full set of MAPK and transcription factors, which might cooperate for the induction of an optimal pro-inflammatory innate immune response.

Another feature of KARAP/DAP12 transgenic mice is the progressive development of a fatal inflammatory syndrome, which occurs after 4 weeks of age, and leads to premature death within 1-2 months, seemingly without any evidence of infection. The nature of the cells and of the cell surface receptors associated with KARAP/DAP12 which are responsible for the onset of the fatal inflammatory syndrome remains to be elucidated. However, the neutrophilia and the lung infiltration with macrophagic type cells that express the transgene suggest that these cells are involved in the pathogenesis of the wasting syndrome. Neutrophilia might be the consequence of excess serum levels of G-CSF and/or TNF-α, but also linked to an effect of TREM-1 engagement on neutrophil survival. Irrespective of these possibilities, the neutrophilia observed in G-CSF transgenic mice is relatively benign in contrast to the wasting disease observed in Tg-hKARAP mice, emphasizing the triggering function of KARAP/DAP12 polypeptides in inflammation. Along this line, cross-linking of TREM-2a on mouse macrophage cell lines leads to the release of nitric oxide, and TREM-1 engagement lead to the marked up-regulation of CD86, CD40, ICAM1, CD83, FcγRII and CD11c on human monocytes. Despite this autonomous function of KARAP/DAP12-associated receptors, the possibility that the wasting syndrome observed in Tg-hKARAP mice does not occur in conjunction with other signals cannot be excluded. In particular, the absence of detectable infection does not formally rule out the development of undetected opportunistic microbial infection.

1. a method for the identification of compounds capable of inhibiting a KARAP-transduced signal, comprising the administration of a test compound to a transgenic animal that expresses functional KARAP in at least 3 copies, preferably more than 4 copies, and more preferably in 5, 11, or 30 copies, the test compound being selected as an inhibitor of KARAP-transduced signal when it significantly increases the life duration of the animal, and/or significantly increases the number of lymphoid cells in the animal, and/or significantly decreases the number of myeloid cells in the animal.

2. A method for the identification of compounds capable of inhibiting a KARAP-transduced signal comprising:
inducing a reaction involving at least KARAP, in animals which express a normal number of copy (i.e. 2 copies) of functional KARAP, these animals being referred to as normal animals,
applying the same reaction protocol to animals which have been engineered so as to bear non functional KARAP, these animals being referred to as knock-in animals,
applying the same reaction protocol to a transgenic animal that expresses functional KARAP in at least 3 copies, preferably more than 4 copies, and more preferably in 5, 11, or 30 copies,
administering the same test compound to the normal, knock-in animals and transgenic animals under same or comparable conditions, the test compound being selected as an inhibitor of KARAP-transduced signal when it inhibits said reaction of the normal animals, and when:
it does not significantly inhibit the reaction that may be observed in knock-in animals in response to said reaction protocol application (if any reaction is observed in these knock-in animals), and/or
it does inhibit said reaction in the normal animals to a level that is comparable to the one observed in the knock-in animals, and/or
it does inhibit said reaction in said transgenic animals,
it does not induce any significant immunological or non immunological side effects in the knock-in animals.

3. A method for the identification of compounds capable of inhibiting a KARAP-transduced immune response, comprising:
a) the bringing into contact of a test compound with a cell co-expressing i) a functional KARAP and a functional receptor that transduces signal through KARAP ii) a functional ζ or γ or ε, and a functional receptor that transduces signal through ζ or γ or ε,
b) the stimulation of this KARAP and this ζ or γ or ε-associated receptor, the test compound being selected as an inhibitor of KARAP-transduced signal when it significantly inhibits the KARAP-transduced signal and does not significantly inhibit the ζ or γ or ε-transduced signal.

4. A method for the identification of compounds capable of inhibiting a KARAP-transduced signal without inhibiting another activatory molecules, comprising the selection of those test compounds which interact with the charged aminoacid of the KARAP molecule transmembrane region and/or with a charged aminoacid (K or R) which is centrally located within the transmembrane region of an activatory receptor.

5. The method of claim 4, further comprising the steps of an over-expression method according to claim 1 and/or the steps of a KARAP-transduced reaction method according to claim 2 or 3.

6. KARAP-inhibiting compounds useful as active agents in the formulation of drugs intended for inhibiting undesired immune responses, and in particular for inhibiting the activity of cells favoring auto-immune or allo-immune reactions as selected according to the method of anyone of claims 1 to 5.

7. The use of the KARAP-inhibiting compounds according to claim 6, for impairing the development and maturation of dendritic cells.

8. The use according to claim 7, for inhibiting the antigenic presentation of dendritic cells, via synthesis inhibition or through inhibition of the migration of dendritic cells.

9. The use according to claim 1 of KARAP inhibiting compounds according to claim 6, for making drugs intended for inhibiting dendritic cell development or maturation.

10. The use of the KARAP-inhibiting compounds according to claim 6, for preparing drugs for the treatment, prevention, palliation of immune response wherein KAR activation has to be inhibited.

11. The use according to claim 10, for the treatment of contact sensitivity.

12. The use according to claim 10, for the treatment of multiple sclerosis.

13. Kits for the implementation of a method intended for the identification of a compound which is capable of inhibiting a KARAP-transduced signal, comprising any combination of at least two elements selected from the group consisting of non functional KARAP, engineered cells co-expressing functional KARAP and ζ or γ or ε, animals bearing a normal copy number of functional KARAP, engineered animals bearing a number of KARAP copy equal to or above 3, engineered animals bearing non functional KARAP.

14. Kit according to claim 14 comprising enginereed animals bearing a normal copy number of functional KARAP further comprising an agent capable of inducing a sensitivity contact reaction in an animal such as DNBF. (2,4-dinitrofluorobenzene), or an agent capable of inducing an auto-immune disease such as pMOG peptide 33-55.
